(19) United States
(12) Patent Application Publication
(10) Pub. No.: US 2022/0218827 A1
(43) Pub. Date: Jul. 14, 2022

(54) DELIVERY OF THERAPEUTIC COMPOUNDS WITH IRON OXIDE NANOPARTICLES

(71) Applicant: Memorial Sloan Kettering Cancer Center, New York, NY (US)

(72) Inventors: Charalambos Kaittanis, New York, NY (US); Jan Grimm, New York, NY (US)

(21) Appl. No.: 17/408,292

(22) Filed: Aug. 20, 2021

Related U.S. Application Data
(63) Continuation of application No. 16/212,308, filed on Dec. 6, 2018, now abandoned.
Application No. 16/212,308 is a division of application No. 14/429,531, filed on Mar. 19, 2015, now abandoned.
(60) Provisional application No. 61/703,833, filed on Sep. 21, 2012.

(51) Int. Cl. A61K 45/06; A61K 49/18; A61K 47/61; A61K 47/69; A61K 47/58; A61B 5/055; A61K 31/4166; A61K 31/4745; A61K 31/506; A61K 31/52; A61K 31/5377; A61K 31/663; A61K 31/704; G01R 33/50; G01R 33/56

(52) U.S. Cl. CPC A61K 45/06; G01R 33/5601; A61K 49/1863; A61K 47/61; A61K 47/6923; A61K 47/58; A61K 47/6929; A61K 47/585; A61B 5/055; A61K 31/4166; A61K 31/4745; A61K 31/506; A61K 31/52; A61K 31/5377; A61K 31/663; A61K 31/704; G01R 33/50; A61K 49/1854

(57) ABSTRACT

The present technology relates to the field of drug delivery. For example, the present technology provides methods of delivering a therapeutic to a cell where the method includes administering to a cancer cell a drug delivery composition. In this exemplary method, the drug deliver composition includes a (super)paramagnetic iron oxide nanoparticle core, where the nanoparticle core includes a coat non-covalently attached to a therapeutic, and the coat includes at least one of poly(acrylic acid), carboxymethyl dextran, and polyglucose sorbitol carboxymethylether.

### CROSS-REFERENCE TO RELATED APPLICATIONS

This application is a continuation of U.S. patent application Ser. No. 16/212,308, filed Dec. 6, 2018, which is a divisional of U.S. patent application Ser. No. 14/429,531, filed Mar. 19, 2015, which is a National Stage Application of PCT/US2013/060662, filed Sep. 19, 2013, which claims the benefit of and priority to U.S. Provisional Patent Application No. 61/703,833, filed Sep. 21, 2012, the entire contents of each of which are incorporated herein by reference.

### GOVERNMENT SUPPORT

This invention was made with government support under grant number W81XWH-12-1-0509 awarded by the Army Medical Research and Materiel Command. The government has certain rights in the invention.

### FIELD

The present invention relates to the field of drug delivery, in particular the delivery of unmodified cargo molecules (such as doxorubicin and Taxol®) using iron oxide nanoparticles as therapeutic delivery agents. Specifically described are methods to entrap cargo (i.e. known therapeutics (drugs) and other types of molecules) into the exterior coating of iron oxide nanoparticles, including iron oxide nanoparticles approved for use in humans. Additionally, methods describe the use of such drug-loaded nanoparticles as therapeutic delivery agents. Further, methods include quantifying and visualizing the amount of cargo molecule loading levels when preparing these therapeutic agents and then quantifying and visualizing the amount of delivery (i.e. unloading) of these cargo molecules from these nanoparticles using compact magnetic relaxometers, common NMR instruments and magnetic resonance imaging (MM) instruments.

### BACKGROUND

The distribution of therapeutics within the body of a patient contributes significantly to the therapeutics' efficacy, toxicity and clearance. Hence, monitoring the localization of drugs and achieving their delivery to disease sites is of major clinical and pharmacological importance. Drug delivery systems include liposomes, albumin-based therapeutics, polymer-conjugated drugs, and ligand-conjugated drugs. However, one limitation of these technologies is that because the drug is in another form, including conjugated (covalently bound) to other molecules, the system must go through clinical trials as a new drug because the drug was chemically altered and/or attached to another entity. Hence, apart from the new molecules that are attached to, the properties of the drugs change due to the addition of new functional groups that can facilitate conjugation. Also, frequently the drugs need to undergo certain modifications (e.g. an enzymatic cleavage) inside the patient's body and/or within the cells to regain their original structure or therapeutic structure to become biologically active in the intended manner. Therefore, a system wherein the therapeutic is not altered is needed for more efficient drug delivery for treatment of patients.

Further, in order to achieve monitoring of the delivery of therapeutics, molecular constructs of drugs conjugated to tracing moieties or encapsulated within nanoparticle carriers were previously developed. The resulting platforms incorporated new bonds between the drug and the tracing agents or nanoparticles resulting in the formation of new functional groups and new forms of the drug molecules as part of these drug conjugates. These new bonds and molecules also resulted in different effects of the drug when released from the conjugates as compared to the result of administering the drug in its original form. In other words, these tracing conjugates underwent further modification in the body of a patient in order to release these drugs, often in a different form then before attachment to a delivery system. Thus the formation of new bonds and functional groups, which are absent from the parental drugs, also resulted in subjecting these tracing and delivery systems to additional scrutiny (and expense due to lengthy clinical trials) by regulatory bodies such as the United States Federal Drug Administration before their release for use as a therapeutic agent.

Therefore there is a need for delivery compositions and methods to achieve drug delivery of unmodified drugs to their biological targets. In particular, these delivery compositions are needed for combined delivery and monitoring of therapeutic compounds.

### SUMMARY

The present invention relates to the field of drug delivery, in particular the delivery of unmodified cargo molecules (such as doxorubicin and Taxol®) using iron oxide nanoparticles as therapeutic delivery agents. Specifically described are methods to entrap cargo (i.e. known therapeutics (drugs) and other types of molecules) into the exterior coating of iron oxide nanoparticles, including iron oxide nanoparticles approved for use in humans. Additionally, methods describe the use of such drug-loaded nanoparticles as therapeutic delivery agents. Further, methods include quantifying and visualizing the amount of cargo molecule loading levels when preparing these therapeutic agents and then quantifying and visualizing the amount of delivery (i.e. unloading) of these cargo molecules from these nanoparticles using compact magnetic relaxometers, common NMR instruments and magnetic resonance imaging (MM) instruments.

The present invention provides a drug delivery composition, comprising, a (superparamagnetic iron oxide nanoparticle core, wherein said nanoparticle core comprises a coat non-covalently attached to a therapeutic. In one embodiment, said coat surrounds said core. In one embodiment, said therapeutic is attached to the outside of said core. In one embodiment, said therapeutic is non-covalently attached to said coat. In one embodiment, said therapeutic is non-covalently attached to said coat surrounding said nanoparticle. It is not intended that the present invention be limited by the type of coating surrounding an iron oxide nanoparticle core. In one embodiment, said coat includes but is not limited to at least one molecule selected from the group consisting of poly(acrylic acid), carboxymethyl dextran, polyglucose sorbitol carboxymethylether and an amine-functionalized molecule. In one embodiment, said coat comprises at least one molecule selected from the group consisting of poly(acrylic acid), polyglucose sorbitol carboxymethylether, and an amine-functionalized molecule. It is not intended that the present invention be limited by the type of therapeutic. Thus in one embodiment, said therapeutic is selected from but not limited to at least one of the group consisting of bone resorption inhibitor, farnesyl diphospate synthase inhibitor, mTOR inhibitor, tyrosine kinase inhibitor, Hsp90 inhibitor, γ-secretase inhibitor, PI3K inhibitor, lipophilic anti-angiogenic peptidomimetic, androgen receptor antagonist, antimetabolite, antineoplastic, alkylating agent and PI3 inhibitor. It is not intended that the present invention be limited by the individual therapeutic. Thus in one embodiment, said therapeutic is selected from but not limited to at least one of the group consisting of doxorubicin, paclitaxel, mTORC1, mTORC2, BCR/ABL, Src, Alendronate, AZD8055, Dasatinib, PU-H71, GSI-34, BKM120, FR230, MDV3100, 5-fluorouracil, cisplatin and BEZ 235. It is not intended that the present invention be limited by the mixtures of said therapeutic. Thus in one embodiment, said therapeutic is selected from but not limited to at least two therapeutics selected from the group consisting of androgen receptor antagonist and PI3K inhibitor, MDV3100 and BEZ 235, antineoplastics, 5-fluorouracil and cisplatin. In one embodiment, said composition has a T1 and T2 such that said T1 and T2 of the therapeutic coated (super)paramagnetic iron oxide nanoparticle is greater than a T1 and T2 of said coated (super)paramagnetic iron oxide nanoparticle without said therapeutic compound.

The present invention provides a method of loading, comprising, a) providing, i) a coated (superparamagnetic iron oxide nanoparticle core, wherein said coated iron oxide nanoparticle core has a coat comprising at least one molecule selected from the group consisting of a poly(acrylic acid), carboxymethyl dextran, polyglucose sorbitol carboxymethylether, and an amine-functionalized molecule, ii) a cargo molecule capable of being attached to said coat; b) adding said cargo molecule solution dropwise without inducing precipitation to said nanoparticle core; c) mixing said cargo molecule with said nanoparticle core under conditions such that said cargo molecule non-covalently attaches to said coat. In one embodiment, said method further comprises a magnetic device for obtaining T1 and T2 of said (superparamagnetic iron oxide nanoparticles. In one embodiment, said T1 and T2 increase as said cargo molecule is attached to said coat. In one embodiment, said method further comprises a vortex mixing device. In one embodiment, said mixing device is vised when adding said cargo molecule to said nanoparticle core. In one embodiment, said cargo molecule is suspended in an organic solvent for loading onto said coated iron oxide nanoparticle. In one embodiment, said cargo molecule is suspended in dimethyl sulfoxide solution. In one embodiment, said (superparamagnetic iron oxide nanoparticles are in solution. In one embodiment, said (superparamagnetic iron oxide core nanoparticle is suspended in distilled water. In one embodiment, said method further comprises a magnetic device for obtaining T1 and T2 of said (superparamagnetic iron oxide nanoparticles. It is not intended that the present invention be limited by the type of magnetic device. Magnetic devices include but are not limited to compact magnetic relaxometers, NMR instruments and magnetic resonance imaging (MM) instruments (devices). In one embodiment, as said cargo is loading onto said coated (superparamagnetic iron oxide nanoparticle both T1 and T2 of said nanoparticle increases. In one embodiment, as said cargo is loading onto said coated (superparamagnetic iron oxide nanoparticle both T1 and T2 of said nanoparticle in solution increases. In one embodiment, said cargo loaded coated (super)paramagnetic iron oxide nanoparticles have increased both T1 and T2 compared to coated (superparamagnetic iron oxide nanoparticles before loading. In one embodiment, said cargo loaded coated (super)paramagnetic iron oxide nanoparticles have increased both T1 and T2 in solution. In one embodiment, as said cargo is unloading from said coated (superparamagnetic iron oxide nanoparticles both T1 and T2 of said nanoparticles decreases. In one embodiment, as said cargo is unloading from said coated (superparamagnetic iron oxide nanoparticles both T1 and T2 of said nanoparticles in solution decreases. In one embodiment, said cargo unloading is related to pH. Thus in a further embodiment, as the pH of a solution decreases, there is an associated increase in unloading of said cargo molecules.

The present invention provides a method of delivering a therapeutic to a cell, comprising, a) providing, i) a composition comprising (superparamagnetic iron oxide nanoparticle core comprising a coat non-covalently attached to a therapeutic, and ii) a cell, and b) administering said composition to said cell, under conditions such that there is a therapeutic effect. It is not intended that the present invention be limited by the type of cell. In one embodiment, said cell is a cancer cell. In one embodiment, said cancer cell is a prostate cancer cell. In one embodiment, said cancer cell is an androgen-receptor-overexpressing prostate cancer cell. In one embodiment, said cancer cell is a prostate adenocarcinoma cell. In one embodiment, as said cell is located in a tumor, i.e. a tumor cell. Thus in one embodiment, said cell is a tumor cell. It is not intended that the present invention be limited by the type of therapeutic effect. Therapeutic effects include reducing the rate of cell division, reducing the rate of cell growth, altering cell function, cell death, reducing growth of tumors, etc. In one embodiment, said cell therapeutic effect is cell death. In one embodiment, said method further provides a magnetic device for obtaining T1 and T2 of said (superparamagnetic iron oxide nanoparticles. Magnetic devices include but are not limited to compact magnetic relaxometers, NMR instruments and magnetic resonance imaging (MRI) instruments (devices). In one embodiment, said method further comprises the step of using said device for obtaining T1 and T2 of therapeutic loaded coated (superparamagnetic iron oxide core nanoparticles before administration to said cell. In one embodiment, said use of said device is in vitro. Thus in one embodiment, said method further includes step b) further using said device for obtaining T1 and T2 as said therapeutic is administered. In one embodiment, said T1 and T2 of said nanoparticles decreases in relation to T1 and T2 obtained before administration. In one embodiment, said T1 and T2 of said nanoparticles is decreased in relation to T1 and T2 obtained before administration. In one embodiment, said decreased T1 and T2 is associated with drug delivery. In one embodiment, said therapeutic is administered in vitro to said cell. Thus in one embodiment, said cell is in solution. In one embodiment, said cell is located in a patient. In one embodiment, said use of said device is in vivo. It is not meant to limit the type of magnetic device. In one embodiment, said magnetic device is a magnetic resonance imaging device. In one embodiment, said method further includes step b) further comprising using said magnetic resonance imaging device for obtaining T1 and T2. In one embodiment, said therapeutic is administered in vivo to said cell. In one embodiment, as said therapeutic is administered T1 and T2 decreases in relation to T1 and T2 before administration. In one embodiment, after said therapeutic is administered T1 and T2 is decreased in relation to T1 and T2 before administration. In one embodiment, said decreased T1 and T2 is associated with drug delivery.

### Definitions

To facilitate understanding of the invention, a number of terms are defined below.

As used herein, “a” or “an” means “at least one” or “one or more.”

As used herein, “theragnostic” refers to a combined therapeutic that delivers a therapeutic agent to the disease site and also serves as a diagnostic system, for example, a therapeutic iron oxide nanoparticle (IONP) of the present inventions which may deliver a drug and also provide an Mill signal showing how much of the drug was delivered.

As used herein, “theranostic” refers generally to a merger between therapeutics and diagnostics, for example, a cell killing test to either predict patient's response to a drug delivered by a loaded nanoparticle of the present inventions performed by removing cancer cells for in vitro testing or selection of patient sub-population for clinical trials by using nanoparticles of the present inventions for imaging sites intended for drug delivery by loaded IONP. In addition to in vitro testing, a theranostic system can be used in vivo, such as in patients or animal models, for example to deliver therapeutics and monitor their homing and localization within the body, to image and monitor response to treatment, and to perform prognosis prior and during treatment. A therapeutic component may comprise of cytotoxic drugs, inhibitors, peptides, peptidomimetics and proteins, among others. The diagnostic component may consist of imaging agents, such as iron oxide nanoparticles, to mediate detection with MRL In other words, a theranostic agent is an in vivo system or a molecular assay or other assay used to deliver therapeutics in the body or cells and provide diagnostic information, including prognosis, response to treatment, drug localization and determination of the optimum dose of the drug for the right person at the right time, i.e. personalized medicine.

As used herein, “payload” or “cargo” or “cargo molecule” or “molecular payload” refers to compound or molecule that is loaded onto a coated nanoparticle of the present inventions. Non-limiting examples include fluorophores, drugs, and chemotherapeutics, such as doxorubicin and Taxol® (Paclitaxel), as well as peptides and peptidomimetics. In some embodiments, cargo molecules are hydrophobic drugs, i.e. therapeutic compounds.

As used herein, “intercalation” or “incorporated” in reference to a cargo molecule loading onto a coated IONP of the present inventions, refers to the reversible inclusion, such as by electrostatic forces (including Van der Waals forces and hydrogen bonding) and hydrophobic interactions, of a molecule (or compound) into other molecules (or groups), i.e. one or multiple molecules of the polymeric coating surrounding an IONP of the present inventions. As opposed to “encapsulation” which refers to a cargo molecule surrounded by another molecule which requires the action of a third molecule, such as an enzyme or ion (for instance in acid-mediated hydrolysis for chemical degradation) of the second molecule that physically confines and surrounds the cargo molecule within it or within supramolecular assemblies of multiple second molecule. Examples of encapsulation include cargo molecules confined by lipids and polymer coatings within internal cavities as the coating is being formed while surrounding and forming a polymeric nanoparticle, cargo molecules incorporated into liposomes as the liposomes are being formed, etc. As also opposed to “conjugation” which refers to a cargo molecule chemically attached to another molecule that requires the action of a third molecule, such as an enzyme or reducing molecule (i.e. glutathione), ion, or cell surface receptor, for release, of a cargo molecule that is attached to it by covalent bonds.

As used herein, “loading” in reference to a coated IONP refers to the addition of a cargo molecule to a solution of coated IONPs for intercalation of the cargo molecule into the shell coating of the IONP forming non-covalent bonds with the shell molecules.

As used herein, “T1 increases” refers to the increase in T1 of loading or loaded coated IONPs as compared to the T1 value of a coated IONP. As used herein, “T2 increases” refers to the increase in T2 of loading or loaded coated IONPs as compared to the T1 value of a coated IONP.

As used herein, “loaded” or “payload-carrying” in reference to a coated IONP refers to a coated IONP where a cargo, such as a drug, was incorporated or intercalated non-covalently into the outer coating of an IONP providing a “loaded coated (super)paramagnetic iron oxide nanoparticle” or “loaded coated IONP”.

As used herein, “non-covalent” in reference to bonding, for example, a “non-covalent bond” refers to a type of chemical bond between molecules that does not involve sharing of pairs of electrons, for examples, non-covalent interactions between molecules include but are not limited to hydrogen bonds, ionic bonds, hydrophobic interactions and van der Waals forces (dispersion attractions, dipole-dipole and dipole-induced dipole interactions). Where molecules are associated “non-covalently” these molecules have non-covalent bonds.

As used herein, “unloading” or “unloaded” or “delivery” refers to a loaded coated IONP wherein the loaded molecules are being released from the outer coating of the IONP, with the IONP remaining structurally intact.

As used herein, “coating” or “outer shell” or “outer coating” or “coated” in reference to an IONP refers to a compound located on the outer surface, i.e. “coat” or “shell” of an IONP. Examples of coatings generally include molecules such as carbohydrates, such as polyglucose sorbitol carboxymethylether, amides, polymers, such as poly(acrylic acid), dextran, derivatives of dextran, such as carboxymethyl dextran, a modified dextran, polyglucose sorbitol carboxymethylether, etc. Coating molecules may have functional groups, including hydroxyl, amine, forming aminated coatings, carboxyl and methyl groups, among others. As used herein, “coated IONP” refers to an IONP with an outer shell capable of incorporating or intercalating a cargo of the present inventions, for example, a polymer coated IONP, a poly(acrylic acid)-coated IONP, an aminated IONP, a carbohydrate coated IONP, etc. The term “core,” as it relates to the nanoparticles referred to herein, refers to the center of a nanoparticle, i.e., when a shell is present, the core is located inside of the shell.

As used herein, “Feraheme®” in reference to an IONP refers to a coated “(superparamagnetic” or “superparamagnetic” iron oxide nanoparticle coated with a carbohydrate shell, i.e. carboxymethyl dextran or polyglucose sorbitol carboxymethylether. Feraheme® particles have an exemplary size range between 17-31 nm in diameter.

As used herein, “(superparamagnetic iron oxide nanoparticle” or “IONP” or “iron oxide nanoparticle” or refers to a nanoparticle consisting of an iron oxide core of magnetite (Fe₃O₄) and/or maghemite (y Fe₂O₃), in other words an “iron oxide nanoparticle core”. In general, an IONP in reference to a loaded IONP refers to an IONP having a coating, with an exemplary size ranging from 10 to 250 nm. A (super) paramagnetic iron oxide nanoparticle loses magnetization when deprived of an external field, but in such a field, they exhibit a much higher magnetic moment. The characteristic strong magnetic susceptibility (in comparison to paramagnetic materials) of superparamagnetic particles at this scale is a consequence of the single crystal nature of the material, which permits the iron atoms of the entire crystal to align with the applied field. Superparamagnetic nanoparticles produce “T2” (i.e. spin-spin) and “T1 ” (i.e. spin-lattice) relaxation effects by altering the spins of associated water's protons, usually corresponding to a high r % 1 r_{\} ratio as T2 is the predominant process that is affected, which results in signal reduction on T2-weighted images (“negative” contrast).

As used herein, “dropwise” in reference to “adding dropwise into a solution” refers to using a pipette or eye dropper to add approximately 10 μi of one solution into a larger amount of another solution.

As used herein, “vortexing” in reference to mixing with a votex unit refers to intense mixing for intercalating the cargo molecules into the outer coating of an IONP. Any device capable of achieving or exceeding loading levels of the IONP of the present inventions are contemplated for use.

As used herein, “administered” or “administering” in reference to a therapeutic or compound refers to any method of providing a therapeutic or compound (i.e. cargo) loaded coated IONP to a cell or tissue in culture or to a patient such that the drug or compound has its intended effect on the cell, tissue or patient. For example, one method of administering cargo loaded IONPs in vivo is by an indirect mechanism using a medical device such as, but not limited to a catheter, applicator gun, syringe (for an injection), etc. A second exemplary method of administering is by a direct mechanism such as, local tissue administration (i.e., for example, extravascular placement), oral ingestion, inhalation, etc. In vitro, one method of administering a loaded coated IONP is by adding a loaded coated IONP to the cell culture medium. In other words, administration of a therapeutic to a cell, tissue, tumor, etc. refers to “drug delivery.”

As used herein, “patient” or “subject” refers to a mammal or animal that is a candidate for receiving medical treatment. For example, a mammal may be a human.

As used herein, “therapeutic” refers to a chemical or compound used to treat a disease or condition, such as cancer. Examples of a therapeutic include but are not limited to a drug, peptide, peptidomimetic, protein, etc.

As used herein, “pharmaceutically effective amount,” “therapeutically effective amount,” “biologically effective amount,” and “therapeutic amount” are used interchangeably herein to refer to an amount of a therapeutic that is sufficient to achieve a desired result, i.e. therapeutic effect, whether quantitative or qualitative. In particular, a pharmaceutically effective amount, in vivo, is that amount that results in the reduction, delay, or elimination of undesirable effects (such as pathological, clinical, biochemical and the like) in the subject.

As used herein, “therapeutic effect” in reference to administration of a therapeutic or drug in vitro refers to a change in cell or tissue viability, such as a change in growth, function, death, etc. A “therapeutic effect” in reference to administration of a therapeutic or drug in vivo refers to improved health of the subject, including but not limited to reduction of tumor growth, reduction of harmful disease symptoms, a change in immune function, a change in cell, tissue, endocrine, or organ function. In other words, a therapeutic effect results in the reduction, delay, or elimination of undesirable effects (such as pathological, clinical, biochemical and the like) in the subject.

As used herein, “therapeutic drug monitoring” refers to determining the relative amount of drug circulating in the blood or located in a specific tissue or area of a human body. As used herein, “cell” refers to a single cell as well as to a population of (i.e., more than one) cells. The population may be a pure population comprising one cell type, such as a population of normal cells or a population of cancer cells. Alternatively, the population may comprise more than one cell type, for example a mixed cell population of normal and cancer cells.

As used herein, a “cell line” refers to cells that are cultured in vitro, including primary cell lines, finite cell lines, continuous cell lines, and transformed cell lines, but does not require that the cells be capable of an infinite number of passages in culture. Cell lines may be generated spontaneously or by transformation.

As used herein, the term “cell culture” or “cultured cells” refers to any in vitro culture of cells. Included within this term are continuous cell lines (e.g. with an immortal phenotype), primary cells, finite cell lines (e.g., non-transformed cells), and any other cell population maintained in vitro, for example, primary tissue.

As used herein, the terms “culture media,” and “cell culture media,” and “cell medium” refer to media that are suitable to support the growth of cells in vitro (i.e., cell cultures). It is not intended that the term be limited to any particular culture medium. For example, it is intended that the definition encompass outgrowth as well as maintenance media. Indeed, it is intended that the term encompass any culture medium suitable for the growth of the cell cultures of interest.

As used herein, “cancer” is a general term for diseases that are characterized by the uncontrolled, abnormal growth of cells. Cancer cells spread locally and can intravasate and spread via the bloodstream and lymphatic system to other parts of the body as metastatic cancer. As used herein, “tumor” refers to an abnormal mass of tissue that results from excessive cell division that is uncontrolled and progressive. It is also called a neoplasm. A tumor may be either benign (not cancerous) or malignant. Cancer cells may be located in a tumor. A cell that is part of a tumor is a “tumor cell.”

As used herein, “magnetic device” or “magnetic imaging device” refers to a device for obtaining magnetic measurements or values of target magnetic compounds, such as IONPs. Examples of such devices include but are not limited to compact magnetic relaxometers, NMR instruments and magnetic resonance imaging (MM) instruments (devices).

As used herein, “MRI” or “magnetic resonance imaging” refers to a diagnostic device (imaging device having the capability of obtaining magnetic measurements in vivo) based upon nuclear magnetic resonance (NMR) signal of protons in water, lipids, proteins, etc. in tissue, through the combined effect of a strong static magnetic field and a transverse radiofrequency-field (also a magnetic field, but oscillatory). The counterbalance between the exceedingly small magnetic moment of a single proton (it is convenient to visualize the magnetic moment associated with a proton as a spinning top with a north and south magnetic pole, bearing in mind that the moment of a proton refers to a “field,” not a dimensioned structure), and the exceedingly large number of protons present in biological tissue, leads to a measurable effect in the presence of large magnetic fields (Pankhurst et al. J. Physics D—Applied Physics 2003, 36: R167-R181 and in a clinical setting, see, Mornet et al. Journal Materials Chemistry 2004, 14: 2161-2175, each of which is herein incorporated by reference in its entirety).

### BRIEF DESCRIPTION OF THE DRAWINGS

FIG. 1 shows an exemplary addition of cargo within the polymeric coating of PAA IONP that caused an increase in A) T2 and B) T1 with respect to the unloaded IONP (Vehicle) ([Fe]=2.5 μg/mL). Payload-carrying Feraheme® also demonstrated increased C) T2 and D) T1 ([Fe]=10 μ§/ηιE). The incorporation of cargo within Feraheme®'s coating resulted in changes on the nanoparticles' relaxivities (E-F). (Mean±SE).

FIG. 2 shows exemplary changes in the T2 signal upon addition of molecular payload to A) poly(acrylic acid)-coated oxide nanoparticles and B) Feraheme® was imaged by MRI. (Vehicle: unloaded nanoparticles, L: low-loading and H: high-loading for the corresponding preparation, [Fe]pAA

[symbol]

FIG. 3 shows an exemplary gradual incorporation of molecules, such as the fluorescent Taxol® derivative Flutaxl, within the nanoparticles' coating which caused increased A) Feraheme®' s Flutaxl -derived fluorescence emission, as well as the nanoparticle suspension's B) T2 and C) T1 signal ([Fe]=10 μ§/ηιI,). The nanoparticles were first dialyzed to remove unloaded compound, followed by fluorescence and relaxation measurements. (Mean±SE). Size distribution of D) cargo-loaded PAA IONP, E) payload-carrying Feraheme®, F) DiR-intercalated into aminated IONP and G) Flutaxl -encasing Feraheme®. (L: low cargo content; H: high cargo content). (Middle horizontal line of a rectangle =the sample's mean diameter; Upper and lower horizontal lines are the boundaries of the nanoparticles' Gaussian distribution.)

FIG. 4 shows exemplary cargo loading of IONP: A) The presence of cargo within the coating of IONP hindered the diffusion of water molecules, affecting the ability of nanoparticles to efficiently dephase water's protons. B) At high D₂O concentrations, the changes on T2 were abrogated, suggesting that the observed increases in T2 during cargo loading occurred upon blockage of water molecules by the entrapped cargo ([Fe] AA IONP⁻2.5 μg/mL). C-D) Diffusion-weighted MRI revealed that the presence of molecular payload within Feraheme®'s polymeric coating affected the diffusion of water molecules [symbol] μg/mL). (ADC: apparent diffusion coefficient) (Mean±SE).

FIG. 5 shows an exemplary clustering of Feraheme® by Concanavalin A (Con A) facilitated A) decreases in T2, but B) increases in T1 ([Fe]=20 μ§/ηιE). (Mean±SE). C) The nanoparticle aggregation was monitored via dynamic light scattering, indicating the formation of extensive nanoparticle assemblies particularly in the presence of free dextran. The horizontal line in the middle of a rectangle indicates the sample's mean diameter, whereas the upper and lower horizontal lines mark the boundaries of the nanoparticles' Gaussian distribution.

FIG. 6 shows an exemplary pH-dependent release of doxorubicin from drug-loaded Feraheme®. As Feraheme® unloaded its cargo in acidified buffers, the A) T2 and B) T1 decreased ([Fe]=10 μg/mL). Similarly, C) the fluorescence emission of the nanoparticles [symbol] (λex=485 nm, λcm=590 nm) that were retained within the dialysis chamber decreased, due to doxorubicin's release to the free fraction found in the chambers' exterior. D) The release of doxorubicin from Feraheme® to the exterior of the dialysis chamber was confirmed by recording the chemo therapeutic's absorbance in the free fraction at 480 nm. (Mean±SE). E) No changes in the nanoparticle size were observed via DLS during cargo release, suggesting structural integrity of Feraheme® in these conditions. F) Stability of unloaded Feraheme® at different pH. (Middle horizontal line of a rectangle=the sample's mean diameter; Upper and lower horizontal lines are the boundaries of the nanoparticles' Gaussian distribution.)

FIG. 7 shows an exemplary differential release of drugs from Feraheme® preparations in serum. T1 me-dependent study monitoring the A) T2 and B) T1 relaxation times at 0.47 T of doxorubicin-loaded Feraheme® in sterile fetal bovine serum ([Fe]=10 μg/mL). The C) T2 and D) T1 times were recorded during release of Flutaxl from drug-loaded nanoparticles ([Fe]=15 μg mL). (Mean±SE).

FIG. 8 shows an exemplary release of some of their cargo within cells, the A) composite image of bright field and fluorescence images of live LNCaP cells treated for 48 h with Doxorubicin-loaded Feraheme®. Doxorubicin fluorescence is shown in red. B) After washing and trypsinization, the harvested cell pellets were imaged with an Odyssey reader at 800 nm to quantify the uptake of DiR-loaded Feraheme® by the cells. Control cells did not receive treatment. The cell pellets were subjected to iron digestion, and revealed that upon release of some of the cargo the C) r₂ and D) r_{\} relaxivities of DiR-carrying Feraheme® were higher than those of the corresponding fully loaded formulation, suggesting that the cargo-induced effect on IONP's magnetic properties was reversible. (Mean±SE).

FIG. 9 shows exemplary killing of target cells: A-C) Drug-loaded Feraheme® induced increased (enhanced) cytotoxicity to the cells in a prostate cancer cell line LNCaP. The cells were treated for 48 h with equimolar chemotherapeutics concentrations of either free or coated IONPs with intercalated drug. ([Doxorubicin]fmal=7.5 μM, [Fluta l]final=2.2 μM, [Alendronate]fmal=5 μM, [BKM120]final=2.5 μM, [BEZ235]fmal=0.1 μM, [MDV3100]final=5.6 μM) D) Doxorubicin-loaded Feraheme® suppressed tumor growth more effectively than doxorubicin. Three IV administrations were made on day 0, 2 and 6 of the study, with the mice euthanized on day 8. Change in tumor volume was with respect to control animals that were treated with 100 μ\hat{ι}, 10% DMSO-containing IX PBS. (Mean±SE).

FIG. 10 shows an exemplary addition of cargo to poly(acrylic acid)-coated IONP (PAA IONP) compromised (altered) the preparations' A) r₂ and B) r_{\} relaxivities. (Mean±SE). (Vehicle: unloaded nanoparticles, L: low-loading and H: high-loading for the corresponding preparation.)

FIG. 11 shows an exemplary cationic IONP that had amino groups on their surface discovered to intercalate with fluorophore DiR, with concomitant increased A) T2 and B) T1 ([Fe]=5 μg/mL). (Mean±SE). (Vehicle: unloaded nanoparticles, L: low-loading and H: high-loading for the corresponding preparation.)

FIG. 12 shows an exemplary A) Feraheme® could carry large amounts of Flutaxl within its polymeric coat. B) The loading of Feraheme® with cargo did not affect the nanoparticles surface charge potential). (Mean±SE).

FIG. 13 shows an exemplary high-speed centrifugation (revolutions per minute: rpm) of doxorubicin-loaded Feraheme® has a differential effect on A) T2 and B) T1. (Mean±SE).

FIG. 14 shows an exemplary Concanavalin A (Con A) induced clustering of Feraheme®, facilitating A) increases in the preparation's r₂ relaxivity, with B) decreases in r (Mean±SE).

FIG. 15 shows exemplary Doxorubicin-loaded Feraheme® which was stable at physiological pH, without significant changes in the solution's A) T2, B) T1 and C) fluorescence [symbol] nm) across the pH range ([Fe]=10 μ/mL). (Mean±SE).

FIG. 16 shows exemplary DiR-loaded Feraheme® in serum that was stable over several days, with the A) T2, B) T1 and C-D) near-infrared fluorescence remaining unaltered ([Fe]=10 μg/mL). (Mean±SE).

FIG. 17 shows an exemplary schematic of cargo loading to iron oxide nanoparticles (IONP) and affects on their magnetic properties (Scheme 1). Addition of molecular payload induced increases in the T2 and T1, contemplated to be due to obstruction of water molecules from the nanoparticles' outer relaxation sphere. However, as the nanoparticles release their cargo, the relaxation times approach those of the empty nanoparticles, as water molecules can freely diffuse within IONP's outer relaxation sphere.

FIG. 18 shows exemplary changes in Feraheme®'s A) spin-spin (Ara) and B) spin-lattice (Δ ri) relaxivities corresponded to the drug's solubility in DMSO. For the changes in spin-spin relaxivity, the linear regression correlation coefficient was equal to 0.90, while for the changes in spin-lattice relaxivity was 0.79. These data provide a way to predict the changes in Feraheme®'s magnetic properties during cargo loading, by knowing the cargo's hydrophobic character. Solubility information was obtained from Selleck Chemicals, which was the supplier of the listed drugs.

FIG. 19 shows exemplary loading of Adrucil and Cisplatin to Feraheme® (FH) induced changes in the nanoparticles' magnetic properties. Changes in the A) spin-spin and B) spin-lattice relaxivities were observed when one or two (Adrucil/Cisplatin) drugs were loaded. (Mean±SE).

FIG. 20 shows exemplary drug-loaded Feraheme® preparations were more toxic to A) the human prostate adenocarcinoma cells LNCaP (LNCaP-wt) and B) androgen-receptor-overexpressing LNCaP prostate cancer cells (LNCAP-AR). Interestingly, Feraheme® co-loaded with Adrucil and Cisplatin was more effective than the free drugs co-administered in both cell lines. (Mean±SE).

FIG. 21 shows exemplary drug-loaded Ferumoxytol induced enhanced cytotoxicity to the prostate cancer cell line LNCaP when compared to the free nonencapsulated drug. The cells were treated for 48 h with equimolar chemotherapeutics concentrations of either free or nanoparticle-encapsulated drug, a) ([Alendronate] fₘₐₗ=5 μM, b) ABT-737, c) [BEZ235]_{final}=0.1 μM, [MDV3100]finai=5.6 μM). (Mean±SE).

FIG. 22 shows exemplary simultaneous delivery of BEZ235 (NVP-BEZ235) (lower scale) and MDV3100 (enzalutamide, Xtandi) (upper scale) with the same Ferumoxytol nanoparticle resulted in higher cytotoxicity than the delivery of free drugs in prostate cancer cells, a) BEZ235 100 nM/MVD3100 2 microM and b) BEZ235 75 nM/MVD3100 1.5 microM.

This showed that Ferumoxytol can be used for combination therapy in cancer, in order to inhibit multiple oncogenic mechanisms. (Mean±SE).

FIG. 23 shows exemplary Ferumoxytol loaded with doxorubicin achieved faster tumor regression than the free drug in a) prostate and b) breast cancer xenografts. Preferential accumulation of the nanoparticles' cargo is shown at the tumors (c and d). (Mean±SE).

FIG. 24 shows exemplary tumor regression, a) Ferumoxytol loaded with Bortezomib (Velcade®) achieved faster tumor regression than the free drug, b) The drug-loaded nanoparticles significantly reduced the volume of small and large tumors. (Mean±SE).

FIG. 25 shows exemplary retention of drugs within Feumoxytol improved their circulation time and lead to higher tumor uptake. Using a radiolabeled drug (¹³¹I-PU-H71) that was loaded within the nanoparticles, results indicated that Ferumoxytol improved the delivery and biodistribution kinetics of PU-H71 , doubling the drug's accumulation to the tumors.

FIG. 26 shows exemplary Ferumoxytol retained its cargo with weak electrostatic interactions, a) In the presence of high salt concentrations, Ferumoxytol does not load with Doxorubicin, b) Ferumoxytol' s size was not significantly affected across the salt concentration of this study, c) Doxorubicin-loaded Ferurnoxuytol released its cargo at pH 6.8, and the nanoparticles were reloaded with the dye DiR, demonstrating that the loading process was reversible.

FIG. 27 shows exemplary loading Ferumoxytol with cargo increased the T2 a) and T1 b)([Fe]=10 μηα\hat{ι}\hat{ι},). The nanoparticles were first dialyzed to remove unloaded compound, followed by relaxation measurements. The incorporation of cargo within Ferumoxytol's coating resulted in changes on the nanoparticles' relaxivities (c-d). (Mean±SE). e) MM phantom images of unloaded and loaded Ferumoxytol, demonstrating that the cargo does not affect the nanoparticles' T2* signal, as opposed to its effect on T2 (iron concentrations: high=10 μg/mL, medium=6 μ/ηιE and low=4 μg/mL).

FIG. 28 shows exemplary cargo in relationship to water molecules, a) Schematic representation of the proposed model that suggested that the presence of cargo within the coating of IONP hindered the diffusion of water molecules, concomitantly affecting the ability of nanoparticles to efficiently dephase water's protons, b) At high D2O concentrations, the changes on (b) T2 and (c) T1 were abrogated, suggesting that the observed increased T2 and T1 during cargo loading occurred upon blockage of water molecules by the entrapped cargo ([Fe]_{P}AA ioNP³² 2.5 μg/mL) rather than an effect exerted by the payload. d-e) Diffusion-weighted MRI revealed that the presence of molecular payload within Ferumoxytol's coating affected the diffusion of water molecules [symbol] (ADC: apparent diffusion coefficient; vehicle: unloaded nanoparticles). (Mean±SE).

FIG. 29 shows exemplary use of T2* values to determine nanoparticle concentration and localization while T2 and T1 may be utilized for the monitoring of drug release, (a-b) shows exemplary cargo did not affect Ferumoxytol's (FH) T2* signal, as opposed to the increase in c) T2 and d) T1. This result suggested the potential use of T2 to determine nanoparticle concentration and localization, while T2 and T1 may be utilized for the monitoring of drug release, (iron concentrations: high=10 \hat{ }ig /mL, medium=6 μg/mL and low=4 g/mL). (Mean±SE).

FIG. 30 shows exemplary in vivo release of the drug from Feraheme imaged with MRI. The loaded drug affected the T2 signal, so as Feraheme was released the drug in the tumor the T2 approached that of the empty nanoparticles (a and c). The T2* signal was unaffected by the cargo, so it was used to report the amount of nanoparticles at the tumor (b and d).

### DETAILED DESCRIPTION

The present invention relates to the field of drug delivery, in particular the delivery of unmodified cargo molecules (such as doxorubicin and Taxol®) using iron oxide nanoparticles as therapeutic delivery agents. Specifically described are methods to entrap cargo (i.e. known therapeutics (drugs) and other types of molecules) into the exterior coating of iron oxide nanoparticles, including iron oxide nanoparticles approved for use in humans. Additionally, methods describe the use of such drug-loaded nanoparticles as therapeutic delivery agents.

Further, methods include quantifying and visualizing the amount of cargo molecule loading levels when preparing these therapeutic agents and then quantifying and visualizing the amount of delivery (i.e. unloading) of these cargo molecules from these nanoparticles using compact magnetic relaxometers, common NMR instruments and magnetic resonance imaging (MRI) instruments.

Several drugs used in the clinic or in advanced clinical trials showed poor water solubility, hence compromising their effectiveness in vivo. These drugs are also rapidly cleared, while their non-specific uptake sometimes triggered adverse side effects.

Therefore, the methods of making and using a drug delivery system of the present inventions produce a final product that overcomes these limitations. Specifically, the use of coated IONPs of the present inventions for therapeutic drug delivery has the following advantages including but not limited to having a greater stability of a diverse array of loaded therapeutics, such as increased their circulation time, greater accumulation and release of cargo (therapeutic) at the tumor's site, etc. Additionally, the use of a nanoparticle vehicle without modifying (i.e. using a non-covalent bond instead of using covalent bonds) the cargo (i.e. drug, peptide, peptidomimetic, dye) results in release of unaltered cargo molecules.

Additionally, during the development of the present inventions, cargo-induced changes in the magnetic properties of iron oxide nanoparticles (IONP) were demonstrated which are contemplated to be useful for monitoring drug release, using IONP and their polymeric coating's ability to sequester its cargo via weak electrostatic interactions. The inventors discovered that polymer-coated IONP were able to carry a plethora of amphophilic cargos, including fiuorophores and chemotherapeutics, such as doxorubicin and Taxol®. Surprisingly, cargo loading induced significant increases in the transverse T2 and longitudinal T1 NMR proton relaxation times, which were independent of the nanoparticles' surface charge. These findings suggested that the associated IONP's r₂ and T_{\} relaxivities decreased, although the nanoparticle size remained unaltered. These changes in the IONP's magnetic properties correlated directly to the amount of cargo loaded within their polymeric matrix's cavities, suggesting that cargo incorporation altered the interaction of water molecules' within the outer layer of the nanoparticle. Recovery of the IONP's baseline magnetic properties were observed during cargo release, which occurred in slightly acidic conditions (pH 6.8 and below) in vitro and within cells, further indicating that the cargo interacts weakly with the IONP's polymeric coat. This reversible system, facilitated by the cargo molecules and obstruction of water molecules with concomitant T2 and T1 decrease upon drug uptake by a cell or tissue, is different than previously described clustering of nanoparticles (Magnetic Relaxation Switch), with opposing signal changes (T2 signal decreases and T1 increases). Also demonstrated herein, was the surprising finding that doxorubicin-loaded on the clinically approved IONP Feraheme® was more effective than free doxorubicin in vitro and in vivo. Further, coated IONPs already in clinical use are contemplated for utilization for improved in vivo delivery of therapeutic payloads without subjecting the drug or the carrier particle to chemical modification, while evaluation of cargo incorporation and payload levels were readily assessed via bench top magnetic relaxometers, common NMR instruments and MM devices.

Delivery of therapeutic compounds using IONPs of the present inventions has significant advantages over the use of other delivery particles for several reasons, in particular the capability of delivering intact (unaltered) drugs, unlike covalently bonded drugs to delivery vehicles (including nanoparticles). IONPs of the present inventions have shell molecules capable of forming non-covalent bonds with therapeutics. In some embodiments, IONPs of the present inventions are nanoparticles already approved by the United States Federal Drug Association for at least one other clinical use. Specifically, as shown herein, a clinically approved formulation of iron oxide nanoparticles (Feraheme®) along with other types of coated IONPs were discovered to effectively intercalate both amphiphilic and hydrophobic molecules. Weak electrostatic interactions facilitated cargo intercalation within the nanoparticles' polymer, causing changes in the magnetic properties of the nanoparticle formulation. The drugs or other payloads preserved their original structure, since no covalent modifications were involved. The cargo intercalation was reversible, allowing its programmable release in slightly acidic conditions and within target cells. Uniquely, this process of drug release was monitored via magnetic relaxation using laboratory and clinical instruments.

Another advantage of using coated IONPs of the present inventions was the ability to monitor loading and delivery by MM (or NMR) diagnostic and benchtop devices as measured by simultaneous changes in the same direction for both T1 and T2, i.e. both increasing or decreasing. This is in contrast to other types of delivery vehicles where such monitoring shows opposite directional changes in T1 and T2, i.e. T1 increasing while T2 decreases, or T1 decreases while T2 increases, such measurements were not directly related to amounts of drug loading and unloading unlike the observations in the present inventions. Thus in some embodiments, cargo loading increased both T1 and T2 while delivery, i.e. unloading, decreased both T1 and T2.

The inventors contemplated applications of these methods of drug delivery using coated IONPs of the present inventions in the management and treatment of cancer, as well as other debilitated conditions, where administration of effective dosages of therapeutics are important. For instance, patients with cardiovascular disease, arthritis and chronic kidney disease among others may benefit. Pharmaceutical and clinical research might utilize this strategy for new therapeutics and monitor their loading and release in therapeutic platforms using magnetic resonance.

### I. Drug Delivery Particles.

The distribution of therapeutics within the body significantly contributes to their efficacy, toxicity and clearance. Hence, effectively delivering drugs to disease sites is of major clinical and pharmacological importance. To achieve this, molecular constructs of drugs conjugated to targeting ligands or encapsulated within nanoparticle carriers were previously developed. The resulting platforms had new bonds, functional groups and molecules, which are absent from the parental drugs, subjecting these systems to further scrutiny by regulatory bodies. Examples of covalently modified drugs or agents for measuring drugs attached to magnetic nanoparticles includes, WO 2007021621, United States Patent Application No. 20110012596 and 20110053174, an example of composite nanoparticles co-encapsulating drugs and imaging agents includes, United States Patent Application No. 20100330368 while references disclosing coated nanoparticles for use with drugs includes Chertok, et al., “Iron Oxide Nanoparticles as a Drug Delivery Vehicle for MRI Monitored Magnetic Targeting of Brain Tumors. ” Biomaterials. 2008 February; 29(4): 487-496, each of which is herein incorporated by reference. As demonstrated herein, iron oxide nanoparticles (IONP) were able to sequester therapeutic agents within their polymeric coating via weak electrostatic interactions, i.e. through non-covalent bonds, and then released these drugs in slightly acidic conditions and intracellularly.

Furthermore, the loading of drugs to IONP changed the magnetic properties of the nanoparticles, providing a novel way of determining drug loading and release. Thus, during the development of the present inventions, polymer-coated IONPs were discovered to be able to carry a plethora of amphophilic cargos, including fluorophores and chemotherapeutics, such as doxorubicin and Taxol®. Surprisingly, cargo loading induced significant increases in the transverse T2 and longitudinal T1 NMR proton relaxation times, thus allowing utilization of a variety of polymer-coated IONP. These findings indicated that the associated IONP's r₂ and r_{\} relaxivities decreased, although the nanoparticle size remained unaltered. Furthermore, the cargo's effect on the IONP's magnetic properties correlated directly to the amount of cargo loaded within the nanoparticles' polymeric matrix's cavities. Recovery of the IONP's baseline magnetic properties was observed during cargo release, which occurred in slightly acidic conditions in vitro (pH 6.8 and below) and in cells. This reversible system, which is associated with both T2 and T1 increases upon intercalation of a drug, was different from the previously described IONP clustering (magnetic relaxation switch) with opposing signal changes (T2 signal decreased while T1 increased).

Further, Feraheme®, a clinically approved IONP, was observed to intercalate various chemotherapeutics, such as doxorubicin and Taxol®. Surprisingly, the drug-loaded Feraheme was more effective than the free drug in vitro and in vivo. Overall, IONP already in clinical use were utilized for improved in vivo delivery of therapeutic payloads without subjecting the drug or the carrier particle to chemical modification. Furthermore, evaluation of cargo incorporation and payload levels were readily assessed via benchtop magnetic relaxometers, common NMR instruments and MRI.

### II. Alternative To Drug Encapsulation.

Drug encapsulation was used for many other types of delivery methods. For instance, liposomal fon vlations of chemotherapeutics, such as doxorubicin (Doxil) and amphotericin B (AmBisome), are in clinical use, due to their improved pharmacokinetics and ability to deliver high loads of drugs with poor aqueous solubility (O'Brien, et al., Reduced cardiotoxicity and comparable efficacy in a phase III trial of pegylated liposomal doxorubicin HC1 (CAELYX/Doxil) versus conventional doxorubicin for first-line treatment of metastatic breast cancer. Ann Oncol 2004, 15(3):440-9; Ringden, et al., Efficacy of amphotericin B encapsulated in liposomes (AmBisome) in the treatment of invasive fungal infections in immunocompromised patients. J Aiitimicrob Chemother 1991, 28 Suppl B:73-82, each of which is herein incorporated by reference in its entirety).

Alternatives to liposomes are polymeric nanoparticles that consist of biodegradable polymers, such as poly(lactic-co-glycolic) acid (PLGA) and hyperbranched polyesters (HBPE), which were degraded in the body through discrete hydrolytic mechanisms mediated by enzymes, such as esterases, and acidic conditions (McCarthy, et al., Polymeric nanoparticle preparation that eradicates tumors. Nano Lett 2005, 5(12):2552-6; Santra, et al., Cytochrome C encapsulating theranostic nanoparticles: a novel bifunctional system for targeted delivery of therapeutic membrane-impermeable proteins to tumors and imaging of cancer therapy. Mol Pharm 2010, 7(4): 1209-22; Santra, et al., Aliphatic hyperbranched polyester: a new building block in the construction of multifunctional nanoparticles and nanocomposites. Langmuir, 26(8):5364-73 (2010), each of which is herein incorporated by reference in its entirety). These nanoparticles were used for the delivery of drugs, such as Taxotere, in cultured cells and animal models, where the encapsulation process resulted in loading of the drugs within the nanoparticles' polymeric cavity and allowed the use of the nanoparticle's surface functional groups for further bioconjugation, i.e. forming covalent bonds. Other examples of a delivery nanoparticles includes iron oxide nanoparticles (polyacrylic acid (PAA)-coated IONPs) with ligand targeting molecules (folate) loaded with TaxolTM and a lipophilic near infrared (NIR) dye used for treating cells in vitro (Santra, et al, “Drug/dye-loaded, multifunctional iron oxide nanoparticles for combined targeted cancer therapy and dual optical/magnetic resonance imaging.” Small, 2009, Aug 17;5(16):1862-8 (online: 20 Apr. 2009)); iron oxide particle cores coated with glyceryl monooleate (GMO paclitaxel, rapamycin or combination of the drug used in treating breast cancer cells in vitro (WO/2010/134087), in addition to other types of drug loaded IONPs, such as iron-oxide core nanoparticles coated with oleic acid (OA) followed by coating with Pluronic F-127 loaded with doxorubicin and paclitaxel (individual or together) for use in drug delivery and MRI (Jain, al., “Magnetic Nanoparticles with Dual Functional Properties: Drug Delivery and Magnetic Resonance Imaging.” Biomaterials 2008 October; 29(29): 4012\hat{ }-021, each of which is herein incorporated by reference in its entirety). Magnetic nanoparticles are even described for imaging concentrated locations of substrate-enzyme interactions (activity) by T1 and T2 changes (United States Patent Application No. 20080305048), herein incorporated by reference in its entirety).

The loaded IONP of the present inventions, wherein the cargo, i.e. therapeutic, was intercalated into the outer shell of the IONP, is contemplated for use in intercalation of therapeutic entities within nanoparticles. This alternative composition confers targetable delivery, aqueous stability and longer circulation times, without subjecting the drug to chemical modification (Blanco, et al., Molecular-targeted nanotherapies in cancer: enabling treatment specificity. Mol Oncol, 5(6):492-503 (2011) ; Prakash, et al., Polymeric nanohybrids and functionalized carbon nanotubes as drug delivery carriers for cancer therapy. Adv Drug Deliv Rev, 63(14-15):1340-51 (2011); Schroeder, et al., Treating metastatic cancer with nano technology. Nat Rev Cancer, 12(1):39-50 (2012), each of which is herein incorporated by reference in its entirety. Although targetable nanoparticles and drugs conjugated with targeting moieties were used to specifically deliver therapeutics to cells, the release of the drug from these entities was mediated by an enzymatic event or chemical modification of the drug delivery vehicle or the drug construct (drug with targeting moiety). Hence, theranostic nanoparticles were developed, with the unmodified drug's cytotoxic potential preserved within the nanoparticles' cargo bays and the nanoparticle utilized for both drug delivery and prognosis via clinical diagnostic modalities.

The magnetic properties of superparamagnetic iron oxide nanoparticles (IONP) were previously used for the development of sensitive assays, due to the nanoparticles' ability to affect the proton nuclear magnetic resonance (NMR) signal of the surrounding water molecules. Specifically, the nanoparticles primarily affect the transverse or spin-spin relaxation time of bulk water protons (T2), facilitating detection of biomolecules as little as a few attomoles or even a few cancer cells in clinical samples, such as blood (Grimm, et al., Novel nanosensors for rapid analysis of telomerase activity. Cancer Res 2004, 64(2): 639-43; aittanis, et al, Rapid and sensitive detection of an intracelmlar pathogen in human peripheral leukocytes with hybridizing magnetic relaxation nanosensors. PLoS One 2012, 7(4):e35326; Kaittanis, et al., One-step, nanoparticle-mediated bacterial detection with magnetic relaxation. Nano Lett 2007, 7(2):380-3; Kaittanis, et al., Rapid nanoparticle-mediated monitoring of bacterial metabolic activity and assessment of antimicrobial susceptibility in blood with magnetic relaxation. PLoS One 2008, 3(9):e3253 (2010); Kaittanis, et al., Emerging nanotechnology-based strategies for the identification of microbial pathogenesis. Adv Drug Deliv Rev, 62(4-5):408-23 (2010); Perez, et al., Integrated nanosensors to determine levels and functional activity of human telomerase. Neoplasia 2008, 10 (10):1066-72. Perez, et al., Magnetic relaxation switches capable of sensing molecular interactions. Nat Biotechnol 2002, 20(8):816-20, each of which is herein incorporated by reference in its entirety).

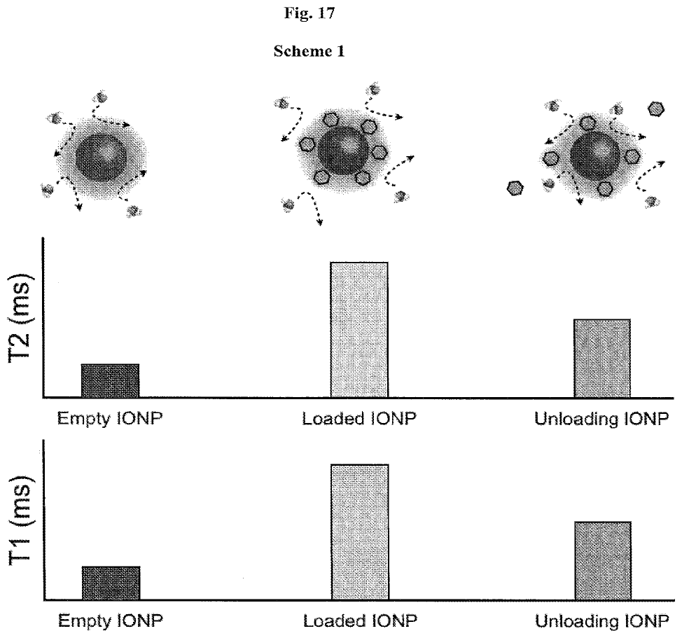

Simple compact relaxometers, NMR or magnetic resonance imaging (MRI) instruments can record changes in the T2 signal, allowing the use of IONP in diverse assays and different environments, ranging from the bench to the clinic. Hence acknowledging IONP's sensitivity and biocompatibility, since they are made out of iron and biodegradable polymers via water-based protocols, the inventors contemplated that intercalation of cargo within the nanoparticles' polymeric coating may affect IONP's magnetic properties. It is contemplated that entrapment of large hydrophobic molecules, such as fiuorophores and chemotherapeutics, within the polymeric coating prevents the diffusion of water molecules within the nanoparticles' outer relaxation sphere (FIG. 17; Scheme 1). This may translate to relaxation time (T2 and T1) increases that were proportional to the amount of molecular cargo carried by the nanoparticles, enabling the quantification of the nanoparticles' cargo load. Furthermore, microenvironmental properties, such as pH, would mediate cargo release, since the payload is not covalently bound to the nanoparticle polymeric coat. Hence, the unloading of IONP's cargo by iron oxide nanoparticles resulted in recovery of the magnetic properties, to baseline levels prior to cargo intercalation (FIG. 17; Scheme 1). Such an environment-responsive self-reporting system is contemplated as ideal for drug delivery applications, since the cargo is not chemically modified and preserves its molecular integrity, while the delivery system sensitively beacons cargo unloading.

As described herein, the effect the cargo exerts onto the magnetic properties of IONP, using different molecular payloads, including fiuorophores and chemotherapeutic agents was measured. The types of nanoparticles used in the compositions of the present inventions included coated nanoparticles, such as poly(acrylic acid)-coated nanoparticles and amine-functionalized nanoparticles; Feraheme®, a clinically approved IONP coated with carboxymethyl dextran or polyglucose sorbitol carboxymethylether, and the like. In initial studies, incorporation of the cargo within the nanoparticles' coating was found to decrease the formulation's 1⁼2 spin-spin relaxivity, which is defined as M″¹ T2″¹ (where M is the nanoparticle molarity). This measurement showed that cargo-loaded nanoparticles less efficiently dephased protons' spins, resulting in increased T1 and T2.

Specifically, regardless of the nanoparticle preparation used, cargo incorporation induced increases in the T2 values, with respect to the same concentration of unloaded nanoparticles. Surprisingly, as the concentration of payload was increased in the preparation, the nanoparticles less efficiently dephased the spins of water protons, concomitantly increasing both their spin-spin (T2) and spin-lattice (T1) relaxation times. Intriguingly, this cargo-dependent behavior was also observed in Feraheme®, with the concentration of cargo modulating the magnetic properties of the formulation.

Nanoparticle characterization revealed that the size of the nanoparticles was not altered during loading of the cargo, with absence of nanoparticle clustering. However, when Feraheme® was clustered in the presence of Concanavalin A, a carbohydrate-binding protein, the solution's T2 decreased, while the T1 value increased as opposed to the changes occurring upon drug loading (both T2 and T1 increase).

It was contemplated that the changes in the IONP's magnetic properties observed upon payload incorporation might have been attributed to obstruction of water molecules from the nanoparticles' vicinity. To test this, excess of deuterium oxide (D2O) abrogated the differences in T2 between cargo-loaded and unloaded nanoparticles, indicating that the molecular payload's effect is directly exerted on water molecules. Additionally, diffusion MRI, which allows the measurement of apparent diffusion coefficient (ADC), revealed that the ADC of cargo-loaded IONP preparations was lower than that of the unloaded IONP, further indicating that that the higher relaxation times, such as T1, might be a consequence of the cargo affecting the free access of water molecules to the nanoparticles' outer relaxation sphere. In fact, after releasing their cargo, the nanoparticles regained their initial magnetic properties, which were associated with decreased T2 and T1 signals. In the case of Feraheme®, the nanoparticles recovered their magnetic character, when the cargo was released either in slightly acidic conditions, such as at pH 6.8 and below, or after long-term incubation in serum to emulate the nanoparticles' behavior in circulation. The changes in the nanoparticles' magnetic properties were monitored with a compact benchtop relaxometer at 0.47 T, as well as Mill operating at higher magnetic field strengths (4.7T).

Overall, as demonstrated herein, (1) cargo loading on multifunctional coated IONPs induced increases the T2 and T1, (2) unloading of the molecular payload is associated with recovery of the nanoparticles' magnetic properties and (3) clinically approved formulations of iron oxide nanoparticles can facilitate the programmable delivery and release of therapeutics, which can be monitored via magnetic resonance. Mechanistically, this behavior was described as the limited ability of water molecules diffusing through the nanoparticles' polymeric coating at the vicinity of their outer sphere, which may be universal when other biomolecules are encapsulated, such as proteins and hormones among others.

### III. Drug Delivery Particles As Theranostics.

One exemplary goal when delivering therapeutic agents, i.e. drugs, is to have a combined therapeutic that both delivers a therapeutic agent to the disease site and also serves as a diagnostic agent. Thus, understanding the distribution and delivery of therapeutic agents are important for drug development and treatment optimization.

Previous delivery systems provided limited delivery information, however in order to overcome this limitation targeted delivery systems were used. For instance, a chemotherapeutic compound would need to stay in circulation for adequate time, avoiding clearance by professional secretion organs, such as the liver and the kidneys which is one limitation of these systems. Furthermore, the drug should effectively accumulate in the pathology (target), with limited or no uptake by other issues, maximizing therapeutic efficacy while minimizing adverse side-effects.

Researchers utilized several innovative strategies in order to provide desired drug delivery effects while overcoming limitations of existing systems. Examples of publications describing these strategies are listed below. However, even with these numerous strategies, there is still a need for a drug delivery composition for more effective delivery while providing a means for monitoring the location and amount of drug delivery in vivo. Previous strategies included the modification of drugs such as doxorubicin with polymers and targeting moieties, in order to achieve their delivery to tumors either via the enhanced permeability and retention (EPR) effect or via the targeting of overexpressed surface markers (Duncan, The dawning era of polymer therapeutics. Nat Rev Drug Discov., 2(5):347-60 (2003); Duncan, Polymer conjugates as anticancer nanomedicines. Nat Rev Cancer, 6(9):688-701 (2006); Huang, et al., Drug-targeting strategies in cancer therapy. Curr Opin Genet Dev, 11 (1): 104-10 (2001); Moses, et al., Advancing the field of drug delivery: taking aim at cancer. Cancer Cell, 4(5):337-41 (2003), each of which is herein incorporated by reference in its entirety). Additionally, apart from transmembrane receptors involved in signal transduction, several research groups have targeted nutrient receptors, such as the folate or transferrin ones, for the targeted delivery of chemotherapies (Qian, et al., Targeted drug delivery via the transferrin receptor-mediated endocytosis pathway. Pharmacol Rev 2002, 54(4):561-87 (2003); Kularatne, et al., Prostate-specific membrane antigen targeted imaging and therapy of prostate cancer using a PSMA inhibitor as a homing ligand. Mol Pharm 2009, 6(3):780-9; Low, et al., Discovery and development of folic-acid-based receptor targeting for imaging and therapy of cancer and inflammatory diseases. Acc Chem Res 2008, 41(1): 120-9; Gaind, et al., Deep-tissue imaging of intramolecular fluorescence resonance energy-transfer parameters. Opt Lett 2010, 35(9):1314-6(2010), each of which is herein incorporated by reference in its entirety). Furthermore, in order to elucidate these new molecular constructs' spatiotemporal profile, beacons were covalently conjugated to them, facilitating detection with clinical modalities, such as Positron Emission Tomography (PET) and fluorescence imaging (Gaind, et al., Deep-tissiie imaging of intramolecular fluorescence resonance energy-transfer parameters. Opt Lett 2010, 35(9):1314-6(2010); Lu, Molecular imaging of HPMA copolymers: visualizing drug delivery in cell, mouse and man. Adv Drug Deliv Rev 2010, 62(2):246-57(2010); Theeraladanon, et al., Rational approach to the synthesis, evaluation, and (68)ga labeling of a novel 4-anilinoquinoline epidennal growth factor receptor inhibitor as a new imaging agent that selectively targets the epidermal growth factor receptor tyrosine kinase. Cancer Biother Radiopharm, 25(4):479-85 (2010); Santra, et al., Cell-specific, activatable, and theranostic prodrug for dual-targeted cancer imaging and therapy. J Am Chem Soc, 133(41):16680-8 (2011), each of which is herein incorporated by reference in its entirety).

Hence, these elegant approaches, apart from delivering the therapeutic agent to the disease site, were also contemplated as diagnostic systems, giving rise to the field of theranostics. Although results were obtained with these molecular theranostics agents in vitro and in vivo, because the parental drugs underwent extensive modifications, including addition of new bonds, functional groups and even entire molecules, such as fluorophores and radioligand chelators, which altered the drugs' in vivo characteristics, these delivery agents need further efficacy and safety tests. Furthermore, previous drug delivery agents that were capable of delivering unmodified drugs, that were approved for use in humans, had little monitoring of actual delivery information.

Thus, the coated nanoparticles of the present inventions are contemplated to overcome these limitations and provide effective theranostics due to the use of noncovalent bonding of drugs to the outer coating of the IONP that also functioned as a contrast agent for Mill. Furthermore, in one embodiment, IONPs of the present inventions are contemplated to deliver one therapeutic compound while providing a diagnostic agent for monitoring loading and then unloading after delivery through the T 1 and T2 measurements of the coated IONPs before loading, during loading, after loading and during and after drug delivery in vitro and in vivo. Even further, in another embodiment, IONPs of the present inventions are contemplated to deliver at least two or more therapeutic compounds while providing a diagnostic agent for monitoring loading and then unloading during and after delivery. Examples of these embodiments are described herein.

### A. Cargo Incorporation Affected The Magnetic Properties Of IONP.

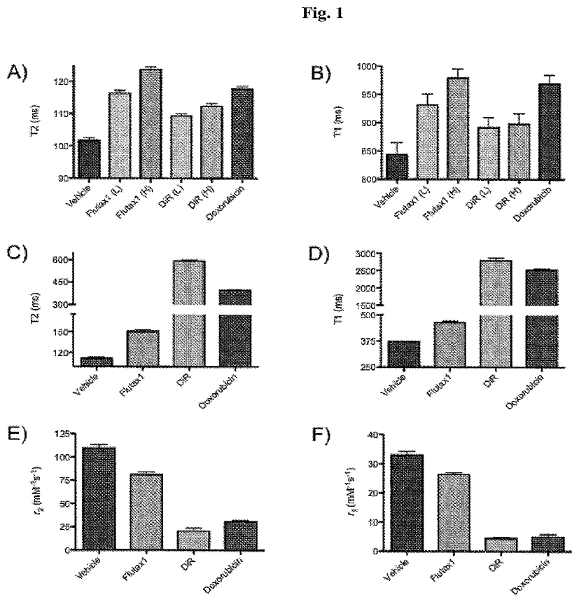

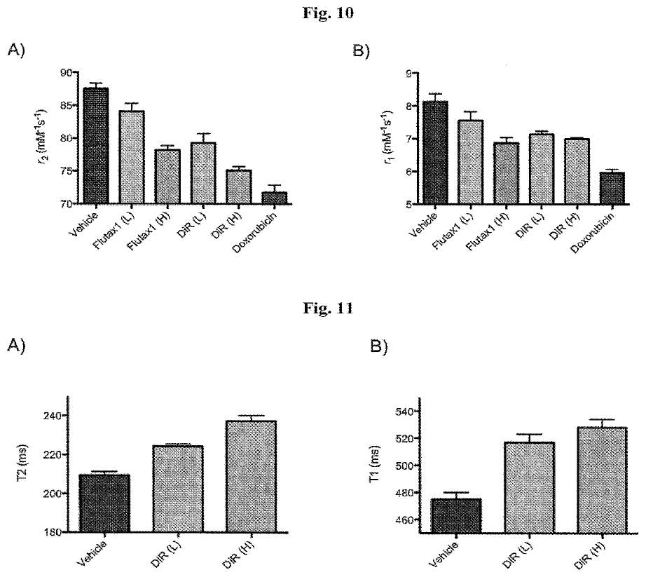

Incorporation of molecular payload on iron oxide nanoparticles increased solution's T2 and T1 relaxation times, contemplated due to displacement of water molecules from IONP's outer relaxation sphere. These relaxation times were measured, first with a poly(acrylic acid)-coated IONP with a variety of different molecular weight cargos, including fluorophores (DiR) and chemotherapeutics (Doxorubicin and Flutaxl; a fluorescent Taxol® derivative). After cargo intercalation within the amphiphilic pockets of the nanoparticles' polymeric coating via the solvent diffusion method, distinct changes were observed in all cargo-carrying preparations, as opposed to the control (vehicle) preparation (FIGS. 1 A-B). Specifically observed was that at the higher concentrations of the payload within the nanoparticles, the more pronounced changes in the relaxation times were observed. For instance, a preparation of IONP with high Flutaxl levels ([FlutaxljiONP⁼30 μM) less efficiently influenced the solution's proton relaxation times than a corresponding preparation with a lower Flutaxl load ([Flutaxl]IONP⁼15 μM). Since T2 and T1 are inversely proportional to a contrast agent's spin-spin (r₂) and spin-lattice r) relaxivities, the high-load Flutaxl-carrying IONP had r₂ and r_{\} relaxivities lower than those of the low-load Flutaxl preparation, indicating that the observed changes were mediated by the amount of cargo within the nanoparticle (FIGS. 10A-B). Similar results were obtained when low and high DiR-carrying IONP formulations ([DiR]iONP⁼1-57 μM and [DiR]IONP⁼5-46 μM) were prepared, with the high-load formulation resulting in higher relaxation times (FIGS. 1A-B, FIGS. 10A-B). Furthermore, intercalation of Doxorubicin increased both the T2 and T1 signal, indicating that nascent chemotherapeutics can be loaded within the nanoparticles and induce changes in the preparation's magnetic properties ([Doxorabicin]iONP⁼12.5 μM) (FIGS. 1 A-B, FIGS. 10A-B). Hence, these results indicated that the changes in T1 and T2 were modulated by cargo incorporation within the IONP's poly(acrylic acid) coating, leading to IONP with compromised (altered) magnetic properties.

### B. Magnetic Properties of IONP Change Upon Cargo Loading.

The inventors subsequently examined whether the change in magnetic properties was observed in other IONP, which were stabilized with other polymeric coatings.

Indeed, aminated IONP behaved similarly to their negatively charged poly(acrylic acid) counterparts, in the presence of molecular cargo (FIGS. 11 A-B). Compared to the corresponding vehicle preparation, the aminated DiR-carrying IONP less efficiently dephased the solution's water protons with concomitant high T2 and T1 readings, although the iron concentration was the same for the three samples. Additionally, these changes were cargo-dependent, as the high-content DiR preparation ([DiR]iONP⁼5-46 μM) facilitated higher T2 and T1 readings than the nanoparticles carrying lower amount of DiR ([DiR] ioNP⁻1-5 μM). Taken together, these results show that loading cargo affects IONP's magnetic properties, regardless of the nanoparticles' polymeric coating and surface charge.

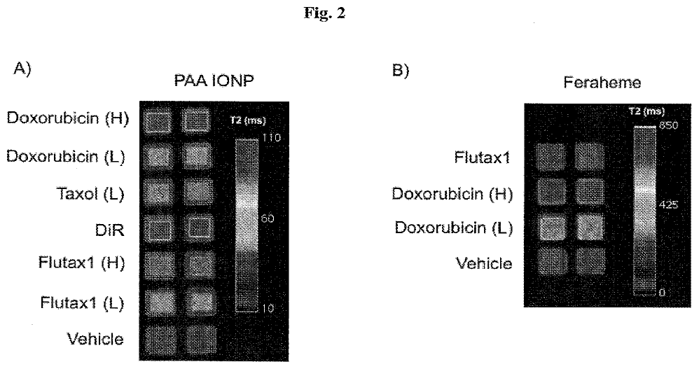

Feraheme® (ferumoxytol), a clinically approved IONP used for the treatment of chronic kidney disease, was tested as an exemplary drug delivery platform for cargo-dependent changes on magnetic properties. Thus, the fluorescent Taxol® derivative ([Flutaxl]Ferahemeg⁼30 μM), doxorubicin ([Doxorubicin]Feraheme®⁼828 μM) and DiR ([DiR]perahemeg⁼920 μM) was loaded onto Feraheme®, which showed cargo-modulated alterations in the T2 and T1 signal (FIGS. 1C-D). These changes were also reflected in the resulting formulations relaxivities. where the r₂ and r_{\} decreased after addition of the cargo (FIGS. 1E-F). Cargo-induced changes in r₂ and r_{\} were also observed when other compounds were loaded into the nanoparticles ([Drug]peraheme®⁼¹00 μM), as well as in a Feraheme® preparation that was co-loaded with the androgen receptor antagonist MDV3100 and the PI3 inhibitor BEZ235 [MDV3100]Feraheme®=250 μM and [BEZ235]Feraheme®=75 μM) (see infra Table 2 in Section IV and under heading “Magnetic Properties of IONP Change Upon Cargo Loading”). This selection of cargo included drugs that have discreet mechanisms of action and targets, which are located at the extracellular matrix and the cytoplasm, as well as at or within the plasma membrane. Since for these experiments a compact magnetic relaxometer was used, MRI was also used to image these changes in the nanoparticles' magnetic signal. Both unloaded suspensions of poly(acrylic acid) IONP and Feraheme® had low T2, while addition of payload gradually increased the T2, with respect to the molecular load of each sample (FIGS. 2A-B).

Hence, it is contemplated that these changes in iron oxide nanoparticles' magnetic signal can be monitored with benchtop relaxometers and commonplace NMR analyzers, as well as animal and clinical MM instruments.

### C. IONP's Molecular Payload Directly Affected The Accessibility Of Water Molecules.

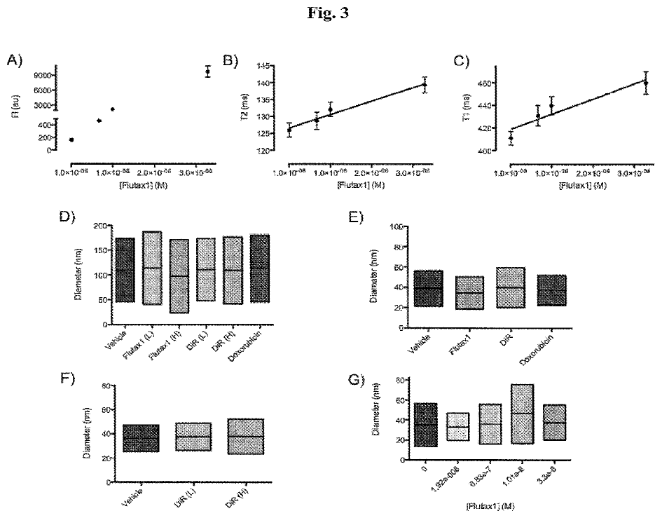

The mechanistic details of this phenomenon were studied from the aspect that payload was non-covalently intercalating within the pockets of the polymeric coating. Thus loading may prevent the interaction of water with the IONP's iron oxide core, by obstructing the access and free diffusion of water molecules from the nanoparticles' outer relaxation sphere. This would reduce the nanoparticles' capability to alter the bulk water's relaxation times. Therefore, increasing amounts of Flutaxl were initially incorporated in the domains of Feraheme®'s coating, while monitoring the preparations' fluorescence emission and magnetic signal. The results showed a correlation of the amount of loaded drug with the observed changes in the relaxation time T1 and T2. Thus, increasing the amount of Flutaxl in the nanoparticles led to an increase of both relaxation T1 and T2 times parallel to an increased fluorescence emission from the loaded Feraheme® (FIGS. 3A-C). Therefore, monitoring T1 and T2 relaxation times should also provide the capability to quantify and monitor the loading of non-fluorescent payloads into IONP.

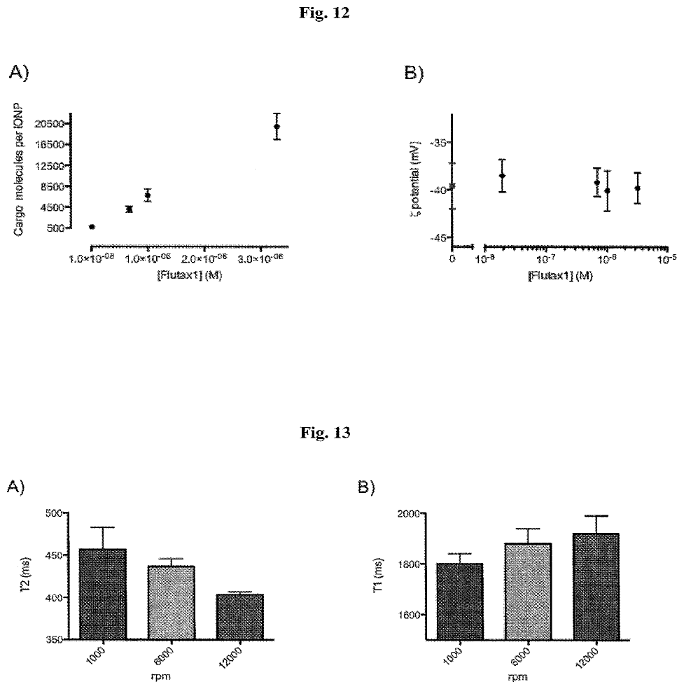

The intercalation process had an apparent loading efficiency of approximately 80%, allowing large amounts of payloads to be intercalated within Feraheme®'s coating without affecting the nanoparticles' surface charge (FIGS. 12A-C). Furthermore, to exclude that the observed changes in the magnetic properties of IONP are caused by changes in the nanoparticles' size distribution, dynamic light scattering (DLS) analysis was performed. This data showed that all cargo-carrying formulations had similar size distributions to that of their corresponding vehicle with the average diameter being unaltered (FIGS. 3D-G), suggesting that the cargo and intercalation process did not structurally change the nanoparticles.

### D. Incorporation of Molecular Payload Within IONP's Coating

### Hindered The Efficient Diffusion Of Water Molecules Through The Nanoparticles' Coating.

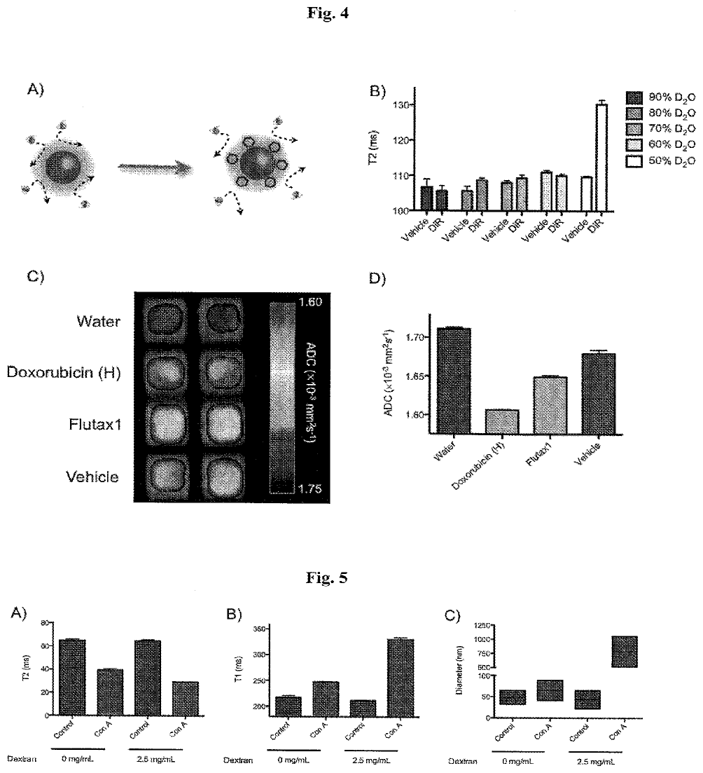

Higher T2 and T1 relaxation times were measured after IONP had molecular payloads (FIG. 4A). Thus to measure that the presence of cargo within the pockets of the coating limits access of bulk water to the proximity of the iron oxide core, a benchtop magnetic resonance reader (Minispec) was used to measure T2 changes of DiR-loaded Feraheme® in solutions with different concentrations of deuterium oxide (D2O). These measurements showed increased T2 after loading indicating the presence of cargo within the pockets of the coating limits access of bulk water to the proximity of the iron oxide core. Since the deuterium nucleus consists of a proton and a neutron, as opposed to hydrogen's single proton, deuterium has a different magnetic moment from hydrogen, allowing its use in the identification of the role of water and hydrogen protons, via nuclear magnetic resonance spectroscopy. Thus as the concentration of D2O increased, the T2 of solutions of DiR-loaded Feraheme® decreased, indicating that as D2O became more abundant in the solution than ¾0, the presence of intercalated cargo was not exerting the initial effect on the water proton relaxation times, due to the low water concentration (FIG. 4B). Further, as the concentration of D2O increased, the T2 of solutions of DiR-loaded poly(acrylic acid)-coated IONP decreased (FIG. 13), which was also attributed to the ability of cargo to obstruct water molecules from the nanoparticles' vicinity, eventually causing T2 increases during cargo loading. These findings were surprising, thus subsequently diffusion-weighted imaging and MRI, were tested in order to deduce if cargo incorporation within Feraheme® impairs the diffusion of water molecules within Feraheme®'s coating, justifying the observed increases in T1. Results emanating from the apparent diffusion coefficient map revealed that the Flutaxl-and doxorabicin-loaded Feraheme® had lower diffusion coefficients than the unloaded Feraheme® (FIGS. 4C-D), supporting that the cargo obstructed the free diffusion of water molecules.

E. T2 Decreased and T1 Increased with Particle Aggregation.

Previous studies used target-induced clustering of IONP in sensitive assays for the detection of numerous biomolecules and targets (Grimm, et al., Novel nanosensors for rapid analysis of telomerase activity. Cancer Res 2004, 64(2):639-43, Perez, et al., Integrated nanosensors to determine levels and functional activity of human telomerase. Neoplasia 2008, 10 (10): 1066-72; Perez, et al., Magnetic relaxation switches capable of sensing molecular interactions. Nat Biotechnol 2002, 20 (8):816-20, each of which is herein incorporated by reference in its entirety) Specifically, it was demonstrated that in the presence of the designated target, the nanoparticles form with increasing target concentration extensive supramolecular assemblies. (Perez, et al., Magnetic relaxation switches capable of sensing molecular interactions. Nat Biotechnol 2002, 20 (8):816-20, oh, et al., Nanoparticle-target interactions parallel antibody-protein interactions. Anal Chem, 81 (9):3618-22 (2009); Kaittanis, et al, The assembly state between magnetic nanosensors and their targets orchestrates their magnetic relaxation response. J Am Chem Soc, 133(10):3668-76 (2011), each of which is herein incorporated by reference in its entirety. Presumably during this extensive nanoparticle cluster formation, water molecules may slowly diffuse within the assemblies, eventually being sequestered from the solution for a substantial time. Hence the multiple outer spheres of the cluster's IONP affect the entrapped water molecules, leading to T2 decreases.

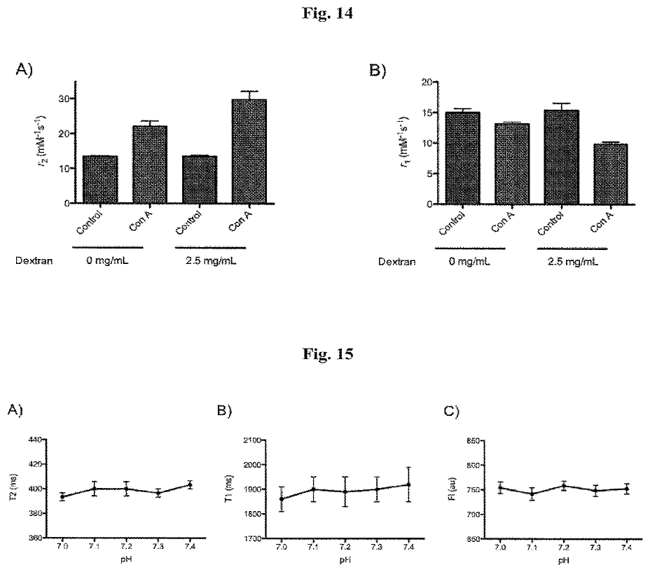

Since this result was different from the observed increases in T2 and T1 during cargo incorporation, T1 was measured for affects during IONP aggregation. Therefore, doxorabicin-loaded Feraheme® formulations were subjected to high-speed centrifugation to induce mechanical aggregation of the nanoparticles. As expected, centrifugation resulted in aggregation through the application of physical force with concomitant decreases in the solution's T2, in line with previous experimental and theoretical work on target-facilitated nanoparticle clustering (FIG. 14A). Also, at higher centrifugal forces the T1 signal increased, indicating that nanoparticle aggregation has a differential effect on the T2 and T1 values of IONP suspensions (FIG. 14B) and that the observed findings after drug loading in the present inventions were not based upon particle aggregation.

To further corroborate these results, Concanavalin A (Con A), a protein that has high affinity towards carbohydrates (Asian, et al., Nanogold-plasmon-resonance-based glucose sensing. Anal Biochem, 330 (1): 145-55 (2004); Yoshizumi, et al., Self-assembled monolayer of sugar-carrying polymer chain: Sugar balls from 2-methacryloyloxyethyl D-glucopyranoside. Langmuir, 15(2):482-488 (1999), each of which is herein incorporated by reference in its entirety) was used to induce the clustering of Feraheme®, since it is coated with a glucose-based polymer. Addition of the lectin ([Con A]fₘₐl=50 g/mL) to IONP ([Fe]=23m/mL) induced decrease in the solution's T2 but increase in the T1 (FIGS. 5A-B), while nanoparticle aggregation was confirmed via DLS (FIG. 5C). Likewise, the differential effect on IONP′s magnetic properties was reflected on their relaxivities, with the r₂ increasing and r_{\} decreasing since they are inversely proportional to the relaxation times T2 and T1 respectively (FIGS. 15A-B).

Moreover, when enhanced nanoparticle aggregation was facilitated in the presence of excess dextran ([Dextran]=2.5 mg/mL), a glucose-based polymer, marked increase in the T1 value was observed, with the T2 decreasing (FIGS. 5A-C) due to clustering of the carbohydrate-coated Feraheme® in the presence of Con A. Taken together, these results demonstrate that IONP aggregation mediated significant decreases in the spin-spin relaxation times (T2), while promoting increases on T1, which become prominent as the aggregates become larger. This may explain why previous studies did not identify an effect of nanoparticle clustering on the T1 process (Taktak, et al., Multiparameter magnetic relaxation switch assays. Anal Chem, 79(23):8863-9 (2007), herein incorporated by reference in its entirety) as the extent of the aggregation state affected the result. The results obtained herein indicated that cargo incorporation and the associated T2 and T1 increases of the present inventions were discerned from nanoparticle clustering and its effect on IONP magnetic properties. Thus it is contemplated that coated IONPs of the present inventions would find use in sensitive characterization of diverse cargo-loaded formulations in research and clinical settings.

F. pH-Dependent Release of Cargo Alters the Magnetic Properties of Loaded IONP.

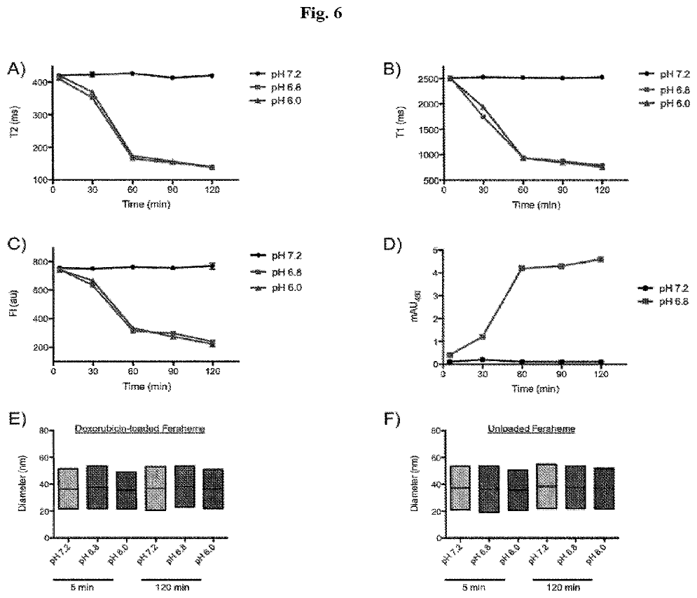

One application for such a system is the use of IONP as a drug delivery platform, which allows for control of loading (and release) by changes in T2 and T1. Ideally, the cargo should be retained at physiological conditions and released in the presence of environmental cues, such as abnormally low pH. This feature is ideal for cancers that exhibit acidic interstitial pH, due to upregulated glycolysis as a result of signaling and metabolic alterations, collectively described as the Warburg effect (Vander Heiden,et al., Understanding the Warburg effect: the metabolic requirements of cell proliferation. Science 2009, 324 (5930), 1029-33, herein incorporated by reference in its entirety). In order to examine the potential use of Feraheme® as a smart drug delivery system, the stability of drug-loaded Feraheme® in phosphate-buffered saline (PBS) at physiologic pH of 7.4 was investigated. Incubation of doxorubicin-loaded Feraheme® in pH-adjusted buffer for 24 hours did not reveal major changes in the T2, and T1 and fluorescence, spanning the pH range encountered during physiological conditions (FIG. 15A-C). However, changes in T2 and T1 were observed at a lower pH of 7.0. Therefore tests were done in order to determine whether the cargo was being released upon acidification of the aquatic milieu. Employing a dialysis chamber to separate the nanoparticles from the potentially released drug, doxorubicin-carrying Feraheme® was incubated in IX PBS adjusted to pH 6.8 and 6.0. Rapid decreases in T2 and T1 were observed in these mildly acidic conditions (FIGS. 6A-B), which were accompanied with decreases in the nanoparticles' fluorescence emission (FIGS. 6C) due to release of doxorubicin as confirmed with HPLC-based spectrophotometry (FIGS. 6D). On the other hand, no changes in T2 and T1 were observed at pH 7.2, likely due to stable accommodation of doxorubicin within Feraheme®'s coating, which is supported by the preparation's unaltered fluorescence emission. To further confirm that these changes were mediated by cargo release, DLS prior and after incubation at these pH levels was preformed. Results indicated that the nanoparticle size and distribution were constant thi ughout the experiment, with the nanoparticle showing little aggregation even at pH 6.0 (FIG. 6E and F). Plausibly, release of cargo at acidic pH may have been facilitated due to disturbance of the weak electrostatic interactions that mediate the association of the drug with the nanoparticles' polymeric backbone secondary to hydrophobic interactions. It is possible that changes in the protonation of the polymer's side chains may disturb hydrogen bonding and van der Waals interaction between the IONP's coating and the cargo, thus triggering release at lower pH. This further demonstrates that the effect of cargo intercalation on IONP is reversible, since payload release facilitates recovery of their magnetic properties, leading to T2 and T1 decreases. Hence, Feraheme® may be utilized for the delivery of chemotherapeutic cargo to the tumor, with the lesion's acidic pH serving as an endogenous trigger for rapid drug release at the tumor's vicinity, maximizing therapeutic efficacy.

G. Effective Feraheme®-based delivery of chemotherapeutics.

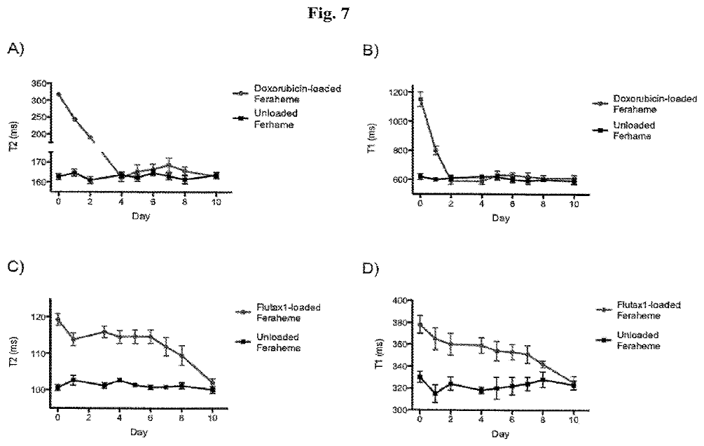

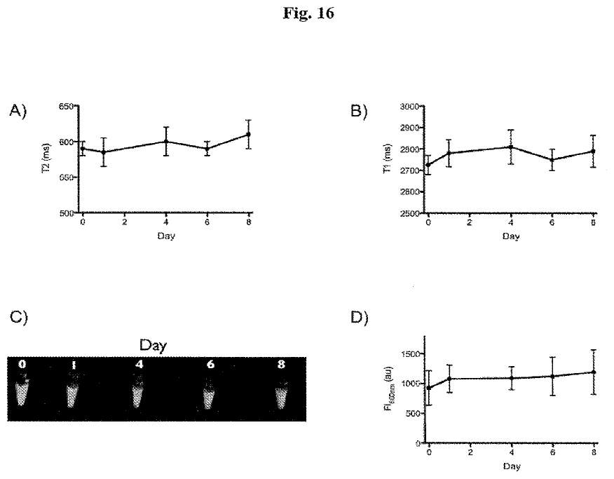

After establishing that cargo release causes decreases in T2 and T1, cargo-loaded IONP was tested for capability to deliver therapeutic payloads within cancer cells. DiR-carrying Feraheme® was found to be stable for up to 8 days in serum (FIG. 16 A-D), whereas doxorubicin- and Flutaxl -loaded nanoparticles released their payload with different kinetic profiles (FIGS. 7A-D). Specifically, Doxorubicin was completely released within less than four days, whereas the fluorescent Taxol® derivative demonstrated sustained release throughout the ten-day duration of the study (approximately 50% drug release after 7 days). These release patterns were attributed to the different physical and chemical characteristics of each payload. For instance, doxorubicin, a small anthracycline DNA intercalator (MW: 580), may be quickly released, due to lower hydrogen bonding formation with the polymer that results in its overall weaker association with the nanoparticles. On the other hand, Flutaxl, a fluorescent microtubule-stabilizing diterpene (MW: 1337), has multiple oxygen and nitrogen atoms that may facilitate its tighter association within the nanoparticle's amphiphilic pockets, as well as multiple segments that favor hydrophobic interactions. Particularly the latter may play a key role in the stabilization of DiR (MW: 1013) within the nanoparticles, due to the presence of two eighteen-carbon-long aliphatic chains. Thus, multiple molecular payloads are contemplated for use in these nanoparticles, targeting several oncogenic pathways. This strategy can potentially achieve the drugs' release at the tumor site while maximizing their circulation time, in a process that is orchestrated by the payload's intrinsic characteristics and initiated by the tumor's aberrant glycolytic activity, without subjecting the drugs to modification.

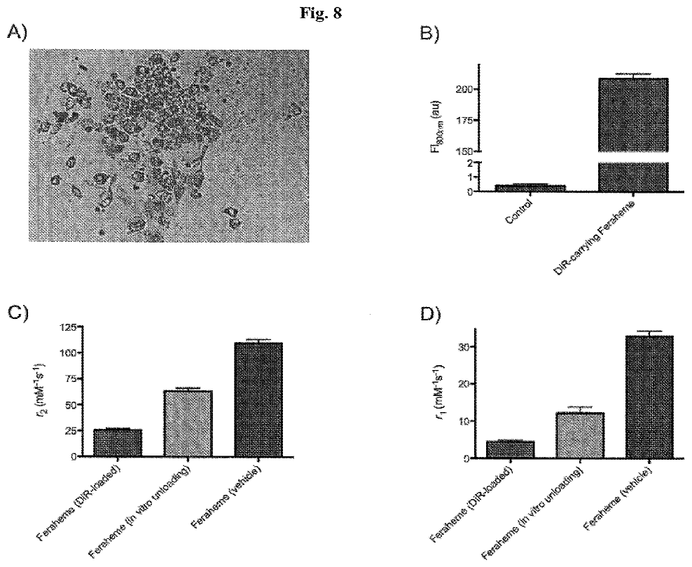

Furthermore, the results shown herein demonstrated that during in vitro cargo unloading by DiR-carrying Feraheme® there was partial restoration of the nanoparticles' magnetic properties that approached those of the original pre-loaded state, where the r₂ and r_{\} relaxivities of the unloading nanoparticles were higher than those of the loaded Feraheme® (FIGS. 8A-B). Apart from measuring the relaxivities of nanoparticles taken up by cells, nanoparticle uptake and cargo delivery were confirmed through fluorescence emission analysis. Cells treated with the DiR-carrying Feraheme® exhibited fluorescence emission at 800 nm, whereas untreated cells or doxorubicin-carrying poly(acrylic acid)-coated IONP had nominal fluorescence (FIGS. 8C). Taken together, these findings indicate that the effects of the cargo on Feraheme®'s magnetic properties are reversible, since payload intercalation relies on non-covalent bonding. Thus, this may facilitate the monitoring of drug loading on Feraheme® and release via magnetic resonance methods, for in vitro and in vivo applications. Particularly for drugs with poor aqueous solubility, this approach can significantly improve their circulation time and stability via a clinically approved nanoparticle-based delivery system. Since the intercalation process was reversible and did not modify the drug chemically, these drugs retained their functional mode of action, exerting their biological effect when released at the acidic tumoric bed or within cancer cells, upon uptake via endocytic processes. Hence, these findings demonstrated that magnetic relaxation are an attractive method for the sensitive characterization of non-fluorescent payloads carried by (superparamagnetic nanoparticles in pharmacological and clinical studies, utilizing portable relaxometers (Haun, et al., Micro-NMR for rapid molecular analysis of human tumor samples. Sci Transl Med, 3 (71):71ral6 (2011); Issadore, et al., Miniature magnetic resonance system for point-of-care diagnostics. Lab Chip, 11 (13):2282-7 (2011), each of which is herein incorporated by reference in its entirety) NMR analyzers and Mill and expanding the applications of IONP in new areas, such as chemoprevention and tumor-directed, personalized therapy.

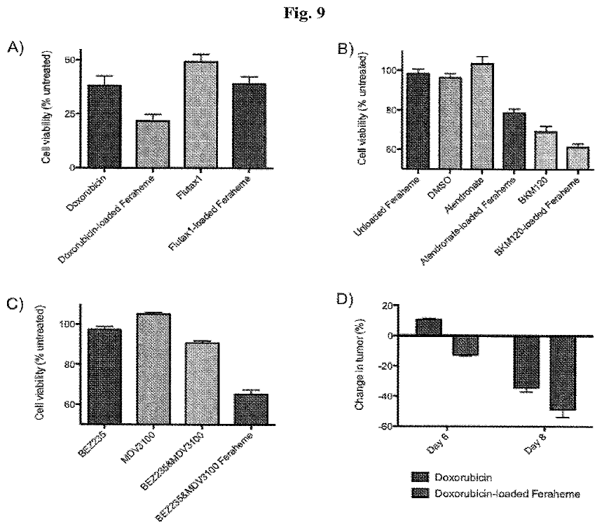

Cargo-loaded iron oxide nanoparticles were tested on cancer cells in order to measure the release of their cargo intracellularly. Control studies demonstrated absence of cytotoxicity in cells treated with unloaded Feraheme®, in accordance with other iron oxide nanoparticle formulations that have different polymeric coatings than this clinically approved agent. Therefore, the in vitro toxicity of Feraheme® carrying chemotherapeutic agents was investigated. Surprisingly, treatment of human prostate adenocarcinoma cells (LNCaP) with drug-carrying Feraheme® resulted in significant cell death, which was more pronounced than treatment with equimolar concentration of the identical free drug (FIG. 9A). For example, doxorubicin-loaded Feraheme® induced more than 75% reduction in cell viability, as opposed to doxorubicin alone that caused a 60% decrease. Similarly, Flutaxl-carrying nanoparticles were 10% more cytotoxic than the drug administered alone, further supporting the notion that delivery of chemotherapeutics with this nanoplatform did not compromise the drug's cytotoxic activity, due to the absence of noncovalent modifications during the intercalation process. Anticancer drugs that target enzymes involved in signal transduction and cholesterol/sterol biosynthesis were tested using methods described herein. The results showed that the loaded-Feraheme® preparations were more effective than free Alendronate and BKM120 (FIG. 9B). The inventors' contemplated that the difference was due to the improved aqueous stability and bioavailability that the nanoparticles conferred to their cargo drug.

Thus in some embodiments, the inventors contemplate administration of a single test dose of loaded coated IONPs having approximately 100 uL of 0.28 mM doxorubicin. In other embodiments, a therapeutic for administration to a patient is contemplated to have approximately up to 1-1000 mM drug concentrations in up to several mis of loaded coated IONPs. In yet other embodiments for combinational therapy, i.e. administration of at least two drugs loaded onto a coated INOP of the present inventions, each therapeutic for administration to a patient is contemplated to have approximately 1 uM up to a 100 mM drug concentration in up to several mis of loaded coated IONPs.

### H. Effective Treatment With Duel Loaded IONP.

Because certain cancers, such as prostate cancer, have more than one pathway involved in oncogenesis and metastasis, Feraheme® was utilized as a drug delivery vehicle for combinatorial therapy. Specifically, since in prostate cancer the crosstalk between the androgen receptor pathway and the PI3K cascade leads to resistance to agents targeting one of the two pathways, (Carver, et al., Reciprocal feedback regulation of PI3K and androgen receptor signaling in PTEN-deficient prostate cancer. Cancer Cell (2011), 19(5), 575-86), herein incorporated by reference in its entirety), developing a strategy that targeted both pathways would be ideal, leading to improved therapeutic efficacy.

Thus, BEZ235, a PI3K inhibitor, and the androgen receptor antagonist MDV3100 were intercalated together into the outer coating of the same IONP, in order to deliver both drugs within prostate cancer cells. The prostate cancer cell line LNCaP was treated with this duel loaded IONP. A prostate cancer cell line LNCaP, which has a functional AR cascade, showed that Feraheme® carrying both BEZ235 and MDV3100 led to more than 30% reduction in cell viability, as opposed to cells treated with one free drug or a combination of both free drugs (FIG. 9C).

Further, in vivo studies revealed that doxorubicin-carrying Feraheme® was more effective than free doxorubicin, after two tail-vein injections into mice of nascent or nanoparticle-loaded doxorubicin (FIG. 9D). Overall, these results show that intercalation of an ampipathic agent within Feraheme® may enhance its bioavailability, preventing its nonspecific association with proteins and lipids, while delivering within tumors and cells at effective dosages.

### I. SUMMARY

During the development of the present inventions, it was discovered and demonstrated that by using compositions and methods described herein, such as the use of multifunctional iron oxide nanoparticles as carriers of amphipathic (therapeutic drugs) and other types of payloads, delivered more effective dosages of drugs to cancer cells than when free drug was administered to cancer cells. Further, the inventors' discovered and demonstrated that magnetic measurements, such as MM, showed reversible changes in both T1 and T2 along with both r_{\} and r₂ that correlated to the amount of cargo loaded onto a coated IONP of the present inventions. Specifically, both T1 and T2 increased (while both r_{\} and r₂ decreased) in proportion to the amount of cargo loaded onto a coated IONP of the present inventions. Conversely, both T1 and T2 decreased (with increasing r_{\} and r₂) in relation to values of fully loaded coated IONPs correlating with loss of cargo, such that the IONPs of the present inventions were used in monitoring drug loading of coated IONPs and then monitoring delivery to cells and tumors, in vitro and in vivo. Therefore, embodiments of the present inventions include comparative T1 and T2 and/or r_{\} and r₂ measurements for duel magnetic imaging results of monitoring drug delivery in combination with diagnostic magnetic imaging of patients.

Therefore, compositions and methods of the present inventions relying on noncovalant bonding i.e. weak electrostatic interactions, such as van der Waals forces and hydrogen bonding, between coating molecules (surrounding IONPs) and drug molecules, preserves the drugs' structure and function, while enhancing their aqueous stability and bioavailability. Specifically, since the drugs resided within the nanoparticles' coating, their circulation time increased significantly, whereas rapid clearance from the kidneys was minimized, due to the nanoparticles' larger size compared to the size of free drug. Additionally, through their residence within the coated iron oxide nanoparticles' shell molecules, the drugs do not directly interact with white and red blood cells or serum proteins.

Therefore, coated IONPs of the present inventions were used for drug carriers (vehicles) for delivering drugs to cells and tumor tissue. Even further, a clinically approved formulation of iron oxide nanoparticles (Feraheme®) was used as a diverse drug delivery platform, capable of accommodating various payloads having a wide range of molecular weights. Therefore, in addition to small drugs, such as doxorubicin, larger agents are contemplated for use as cargo molecules intercalated into coated IONPs, for example within Feraheme®'s polymeric bays. Examples of larger agents for use as cargo molecules in coated IONPs of the present inventions include peptides and peptidomimetics, such as the cyclical FR230 that is an analog of the anti-angiogenic peptide Cilengitide currently used in clinical trials, and compounds having sizes comparable to those of Flutaxl and DiR. Because the process of cargo incorporation was associated with alterations in the nanoparticles' magnetic properties, the loading of non-fluorescent agents was evaluated with magnetic relaxation. Therefore in a research or clinical setting, formulations with different loads of a therapeutic are contemplated for preparation as tailored towards the individual patients' needs, where drug quantification would be quickly determined through magnetic resonance devices. In view of findings described herein, i.e. cargo intercalation resulted in increased T2 and T1 the sensitive quantification of the drug load was achieved, as well as characterization of the release kinetics. The increase in T1 and T2 is contemplated to be due to hindrance of the diffusion of water molecules within the nanoparticles' coating. It was further contemplated that as the amount of cargo intercalating within the nanoparticles' coating, increased the effect on the relaxation times would be more pronounced, due to the interaction of multiple proximal polymer side-chains and functional groups with the cargo molecules. As demonstrated herein, the increased T1 and T2 values associated with cargo molecules loading onto coated IONPs, was different than target-induced clustering, where T2 decreased and T1 increased. Previous studies were not able to identify changes in T1 during nanoparticle clustering, likely due to the strong 1⁼2 relaxivity of the IONP utilized, which was significantly higher than 1⁼\. In addition to in vitro characterization, multifunctional IONP hosting chemotherapeutics in their cavities and radioactive tracers on their surface may facilitate the in vivo assessment of the nanoparticles' localization and drugs' homing with positron emission tomography and MRI. Apart from longer circulation time, the nanoparticles may accumulate and release their therapeutic payload at the tumoric lesion through tumor-intrinsic features, such as its enhanced permeability and retention and Warburg-effect-attributed acidic microenvironment. Furthermore, considering the latest advancements in imaging analysis, multimodality and instrumentation, such as subcellular organelle mapping, hybrid PET/Cerenkov luminescence and intraoperative imaging, (Bhirde, et al., Nuclear Mapping of Nanodrug Delivery Systems in Dynamic Cellular Environments. ACS Nano 2012; Holland, et al., Intraoperative imaging of positron emission tomographic radiotracers using Cerenkov luminescence emissions. Mol Imaging, 10(3):177-86, 1-3 (2011); van Dam, et al., Intraoperative tumor-specific fluorescence imaging in ovarian cancer by folate receptor-alpha targeting: first in-human results. Nat Med, 17(10): 1315-9 (2011), each of which is herein incorporated by reference in its entirety) it is contemplated that iron oxide nanoparticles would be useful in vitro and in vivo, as a self-reporting surrogate drug delivery platform in diverse pathological states and clinical conditions, while providing clinicians with refined monitoring, prognosticating and quantifying capabilities during the course of treatment. Methods of use of the present inventions, including the reversible, non-covalent association of therapeutic compound with an imaging agent, i.e. intercalation of the therapeutic with the nanoparticles' coating facilitates drug delivery and traceability, is contemplated for use by researchers and regulatory agencies (Hamburg, et al., Science and regulation. FDA's approach to regulation of products of nanotechnology. Science, 336(6079):299-300 (2012), herein incorporated by reference in its entirety). Additional exemplary therapeutics for use in the present inventions include those listed in Table 1 below.

TABLE 1
Chemotherapeutics encapsulated by Ferumoxytol.

| Compound | MW | Mechanism of Action | Current Status |
|---|---|---|---|
| Alendronate | 325 | Bone resorption | Osteoporosis treatment and |
| | | inhibitor, | prophylaxis; Phase |
| | | farnesyl diphosphate | III trial for childhood |
| | | synthase inhibitor | cancer survivors |
| Bortezomib | 384 | 20S proteasome | Treatment of multiple |
| | | inhibitor | myeloma and relapsed |
| | | | mantle cell lymphoma |
| Elesclomol | 400 | Oxidative stress | Phase II for ovarian, |
| | | inducer | fallopian tube, and primary |
| | | | peritoneal cavity cancer |
| AZD8055 | 466 | mTOR inhibitor | Phase I trial for recurrent |
| | | (mTORC1, | gliomas |
| | | mTORC2) | |
| Dasatinib | 488 | Tyrosine kinase | Treatment of chronic |
| | | inhibitor | myeloid leukemia |
| | | (BCR/ABL, Src) | and acute |
| | | | lymphoblastic leukemia; |
| | | | Phase II for glioblastoma |
| | | | multiforme, breast, |
| | | | pancreatic, and |
| | | | prostate cancer |
| PU-H71 | 512 | Hsp90 inhibitor | Phase I trial for solid |
| | | | tumors and lymphoma |
| GSI-34 | 534 | γ-secretase inhibitor | Preclinical trials for |
| | | | Alzheimer's and |
| | | | colon cancer |
| Doxorubicin | 580 | DNA topoisomerase | Treatment of |
| | | inhibitor | several cancers |
| BKM120 | 580 | PI3K inhibitor | Phase I and II for advanced |
| | | | solid tumors |
| FR230 | 687 | Lipophilic | Preclinical trials in |
| | | anti-angiogenic | angioplasty |
| | | peptidomimetic | |
| ABT-737 | 813 | BH3 mimetic | Ex vivo evaluation in |
| | | inhibitor of Bcl-xL, | ovarian cancer |
| | | Bcl-2, and Bcl-w | |
| Lapatinib | 925 | EGFR and HER2 | Treatment of breast cancer; |
| | | inhibitor | Phase II for prostate and |
| | | | pancreatic cancer |
| Everolimus | 958 | mTOR inhibitor | Treatment of breast cancer; |
| | | | treatment of kidney cancer; |
| | | | clinical trials for several |
| | | | cancers |
| MDV3100 | 464 | Androgen receptor | Treatment of prostate cancer |
| BEZ235 | 470 | antagonist | Phase II for renal cell |
| | | PI3K inhibitor | carcinoma, breat, prostate, |
| | | | and pancreatic cancer |
| Adrucil | 130 | Thymidylate | Treatment of colorectal and |
| | 300 | synthesis | breast cancer among others |
| | | inhibitor | |
| Cisplatin | 300 | DNA synthesis | Treatment of many cancers |
| | | inhibitor | |

### IV. Exemplary Materials And Methods With Examples.

The following are exemplary materials and methods used during the development of the present inventions in addition to examples.

### A. Materials.

Chemicals were of analytical grade unless otherwise stated herein. Ferrous and ferric chloride salts (FeCl2.4H2O and FeCl3.6H2O) were obtained from Fluka while deuterium oxide (D2O) was from Acros (Pittsburgh, Pa. United States). Poly(acrylic acid) (PAA, MW 1.8 kDa), ammonium hydroxide, hydrochloric acid and dimethyl sulfoxide (DMSO) were obtained from Sigma-Aldrich. Dextran (10 kDa) was acquired from Pharmacosmos (Holbaek Denmark), while the carbohydrate-binding lectin Concanavalin A (Con A) was purchased from Sigma-Aldrich. Payload included but was not limited to the following compounds: Alendronate (MW: 325) from Sigma Aldrich, AZD8055 (MW: 466) from Selleck Chemicals, BEZ235 (MW: 470) from Cayman Chemicals, BKM120 (MW: 580) was a generous gift from Professor Lewis Cantley (Harvard Medical School, Beth Israel Deaconess Medical Center), Dasatinib (MW: 488) from Selleck Chemicals, DiR (1, -dioctadecyl-3,3,3 3′-tetramemylindo-tricarbocyanine iodide, MW: 1013) from Invitrogen, doxorubicin (Adriamycin, MW: 580) from Selleck Chemicals, Flutaxl—a fluorescent Taxol® derivative (MW: 1337) from Tocris Bioscience, FR230 (MW: 687) was kindly provided by Dr. Horst Kessler (Technische Universitat Mimchen), GSI-34 (MW: 534) was provided by Dr. Yueming Li (MSKCC), MDV3100 (MW: 464) was generously provided by Professor Charles Sawyers (MSKCC) and PU-H71 (MW: 512) was provided by Dr. Gabriela Chiosis (MSKCC). Stocks of these chemicals were prepared in DMSO, and stored at −20° C. until further use.

Commercially available iron oxide nanoparticle preparations such as NH2-nanomag®-D-spio and Feraheme® were obtained from Micromod Partikeltechnologie GmbH (Rostock, Germany; NH2-nanomag®-D-spio) and AMAG Pharmaceuticals (Lexington, Mass.; Feraheme®).

### B. Preparation of Iron Oxide Nanoparticles.

Poly(acrylic acid)-coated iron oxide nanoparticles were prepared with the alkaline precipitation method, involving the rapid mixing of ferrous and ferric chloride in ammonium hydroxide that was followed by addition of the polymer solution (for example, see, Santra, et al., Drug/dye-loaded, multifunctional iron oxide nanoparticles for combined targeted cancer therapy and dual optical/magnetic resonance imaging. Small, 5(16): 1862-8 (2009), herein incorporated by reference in its entirety). To remove excess reagents and byproducts, the nanoparticles were washed, concentrated and reconstituted in pH 7.4 phosphate buffered saline (PBS), with a KrosFlo Research II TFF system that was equipped with a 10 kDa column (Spectrum). The nanoparticles were stored at 4° C. until further use, without precipitation being observed, similar to the aminated nanoparticles obtained from Micromod that were used without additional preparation. Feraheme® was subjected to magnetic separation using a IX PBS-equilibrated LS25 MACS column (Miltenyi), in order to isolate IONP with good magnetic properties from free polymer in the solution. Subsequently, Feraheme® was stored at 4° C. until further use.

C. Drug loading into Iron Oxide Nanoparticles.

Intercalation of the molecular payload was achieved using a modified solvent-diffusion-based protocol, facilitating the entrapment of hydrophobic molecules within IONP's polymeric coating. In general, the nanoparticles (25 _{I}A of either PAA-IONP or NH2-nanomagg-D-spio, 30 μ\ddot{υ} of Feraheme®) were resuspended in 500 μ\acute{ι}-\acute{α}\acute{ι}β\acute{ι}\acute{ι}\hat{ι}\hat{ι}ε\acute{α} water, whereas the cargo was diluted to the desired concentration in DMSO (final volume of payload solution was 100 μ\ddot{υ},). The fluorophore or drug payload solution was added dropwise to the nanoparticle solution under vortexing (1000 rpm) at room temperature, without visible precipitation. Subsequently, the preparation was subjected to dialysis in a small-volume dialysis chamber (MWCO 3000, Fisher) against IX PBS. The cargo-carrying IONP were subsequently stored in the dark at 4° C., until further use.

D. Nanoparticle characterization.

Size of IONPs were determined through dynamic light scattering (DLS) (Nano-ZS, Malvern, Westborough, Mass. (MA), United States). The same instrument was used in nanoparticle surface charge measurement (ζ potential), whereas to determine Feraheme®'s nanoparticle concentration the NS500 instrument was utilized (NanoSight, Duxbury, Mass.). Magnetic relaxation measurements, including r and r₂ relaxivities, were determined with a 0.47 T mq20 NMR analyzer (Minispec, Bruker, Billerica, Mass.). For T2 measurements a CPMG pulse-echo train with a 1.5 ms interpulse spacing was used, whereas the T1 sequence varied the interpulse spacing from 5 ms up to 8500 ms. The preparations' iron concentration was determined spectrophotometrically as previously reported (Nath, et al., Synthesis, magnetic characterization and sensing applications of novel dextran-coated iron oxide nanorods. Chem Mater, 21(8):1761-1767 (2009), herein incorporated by reference in its entirety) using a SpectraMax M5 instrument from Molecular Devices. Fluorescence emission measurements were performed using the SpectraMax M5, as well as an Odyssey near-infrared imaging station (LI-COR Biosciences), equipped with two solid-state lasers for excitation at 685 and 785 nm. To determine the cargo load of each preparation, the following molar extinction coefficients were used: ε Doxorubicin=11500 M″¹ cm″¹ at 480 nm, ε Flutaxl=52000 M″¹ cm″¹ at 495 nm and SDiR=270000 M″¹ cm⁴ at 748 nm. Stability experiments were performed in pH-adjusted phosphate buffered saline, whereas serum experiments were performed at 37° C., using fetal bovine serum obtained from Gemini Bio-products. Release of doxorubicin from drug-loaded Feraheme® was performed using a dynamic dialysis setup, as previously described (Santra, et al., Aliphatic hyperbranched polyester: a new building block in the construction of multifunctional nanoparticles and nanocomposites. Langmuir, 26(8):5364-73 (2010), herein incorporated by reference in its entirety). A dialysis chamber was utilized (MWCO 3000, Fisher), containing doxorubicin-loaded Feraheme® in either pH 7.2 or pH 6.8 IX PBS. The nanoparticles were dialyzed against the corresponding pH-adjusted buffer at room temperature and under constant stirring (150 rpm), where at regular time intervals aliquots from the external aqueous milieu of the device were collected for further analysis. The collected samples were analyzed via a Beckman Coulter HPLC instrument, equipped with a CI 8 reverse phase column and set to monitor doxorubicin's absorbance at 480 nm mixing An animal MM from Bruker Biospin operating at 4.7 T and a 35-mm radiofrequency coil were used to image phantoms of the nanoparticle preparations that were spotted on a microplate.

In vitro drug release from loaded IONP. LNCaP cells were grown to confluence, on a 12-well poly(lysine)-coated plate in 10% FBS-containing RPMI medium at 37° C., 5% CO₂. The medium was aspirated, and the cells were supplemented with 1 mL fresh media, plus 50 of either empty (vehicle), Doxorubicin-loaded Feraheme® or DiR-loaded Feraheme®. After 48 h, the cells treated with Doxorubicin-loaded Feraheme® were examined under a Nikon Eclipse T1 fluorescence microscope, in order to determine the nanoparticle uptake. Likewise, following 48 h-long incubation at 37° C., 5% CO₂, the cells treated with vehicle and DiR-loaded nanoparticles were trypsinized and subjected to centrifugation at 1000 rpm for 6 min. The resulting pellets were then resuspended in 400 μ\hat{ι}. IX PBS and aliquoted in two eppendorf tubes for fluorescence emission and magnetic relaxation measurements, using the near-infrared imager (LI-COR) and the benchtop relaxometer (Bruker). For near-infrared fluorescence, excitation was achieved at 785 nm, with emission recorded at 800 nm; with the instrument settings set as follows: focus offset=4 mm, intensity −0.5 and resolution=169 μιτι. The iron content of the cell pellets was determined as described above, with untreated samples of equal cell numbers serving as control.

Cell viability and in vivo studies. LNCaP cells were seeded on black-walled, clear bottom 96-well plates at a cell density of 10,000 cells per well, supplemented with 100 iL 10% FBS-containing RPMI medium. After 24 h growth at 37° C., 5% CO₂, the cells were treated with 10 per well of either free or intercalated drug ([Doxombicin]fₘₐₗ=7.5 μM, [Flutaxl] _{fma}2.2 μM, [Alendronate] _{fina}i=5 μM, [BKM120]_{fma}i=2.5 μM, [BEZ235]_{fina}i=0.1 μM, [MDV3100]_{fmal}=5.6 μM), followed by 48 h incubation (37° C., 5% CO₂). Controls included cells incubated with unloaded nanoparticles or DMSO, corresponding to the free drug's final solvent concentration. Subsequently, the old medium was aspirated, and cell viability was assessed via the Alamar Blue method (Invitrogen). Briefly, the cells were supplemented with 10%-alamar-blue-containing medium (10% FBS-containing RPMI), followed by 3 h incubation in a humidified incubator (37° C., 5% CO2) and recording of fluorescence emission (λ [symbol] =565 nm) with the SpectraMax M5 plate reader. Nude, male mice (n=10) bearing PC3 tumors on their flanks were treated on day 0, day 2 and day 6 with 100 μ\hat{ι}, either doxorubicin alone or doxorubi tin-loaded Feraheme®, both at a final doxorubicin concentration of 0.28 mM. Changes in tumor size were evaluated with a microcaliper, and at the end of the 8-day study the mice were euthanized, according to the Institutional Animal Care and Use Committee guidelines.
Data Analysis. Experiments were performed in triplicate unless otherwise stated, with the results are presented as mean±SEM. The data were analyzed in Prism (GraphPad Software), whereas the MR images were processed through the OsiriX DICOM viewer.
Cargo incorporation affects the magnetic properties of IONP.
Incorporation of molecular payload on iron oxide nanoparticles was contemplated to have facilitated increases in solution's T2 and T1 relaxation times, likely due to displacement of water molecules from IONP's outer relaxation sphere. In order to test whether intercalation of cargo onto coated IONPs, poly(acrylic acid)-coated IONPs and a variety of different molecular weight cargos were tested, including fluorophores (DiR) and chemotherapeutics (Doxorubicin and Flutaxl ; a fluorescent Taxol® derivative). After cargo intercalation within the pockets of the nanoparticles' polymeric coating via the solvent diffusion method, distinct changes were observed in all cargo-carrying preparations, as opposed to the control unloaded (vehicle) IONP preparation (FIGS. 1 A-B). Specifically, the higher the concentration of the payload within the nanoparticles, the more pronounced the changes in the relaxation times were observed. For instance, a preparation of IONP with high Flutaxl levels ([Flutaxl]K>NP=30 μM) less efficiently influenced the solution's proton relaxation times than a corresponding preparation with a lower Flutaxl load ([Flutaxl]IONP=15 μM). Since T2 and T1 are inversely proportional to a contrast agent's spin-spin (r₂) and spin-lattice (n) relaxivities, the high-load Flutaxl-carrying IONP had r₂ and r_{\} relaxivities lower than those of the low-load Flutaxl preparation, indicating that the observed changes are mediated by the amount of cargo within the nanoparticle (FIGS. 10A-B). In line with these findings, similar results were obtained when low and high DiR-carrying IONP formulations were tested, [symbol] μM and [DiR]_{IONP}=5.46 μM, respectively), where the high-load formulation resulting in higher relaxation times (FIGS. 1A-B, FIGS. 10A-B).

Furthermore, intercalation of doxorubicin increased both the T2 and T1 signal, indicating that nascent chemotherapeutics can be loaded within the nanoparticles and induce changes in the preparation's magnetic properties [symbol] μM) (FIGS. 1A-B, FIGS. 10A-B). Hence, these results suggest that this phenomenon is modulated by cargo incorporation within the IONP's poly(acrylic acid) coating, leading to IONP with compromised magnetic properties.

### Magnetic Properties Of IONP Change Upon Cargo Loading.

The inventors subsequently examined whether the change in magnetic properties was observed in other IONP, which were stabilized with other polymeric coatings. Indeed, aminated IONP behaved similarly to their negatively charged poly(acrylic acid) counterparts, in the presence of molecular cargo (FIGS. 11A-B). Compared to the corresponding vehicle preparation, the aminated DiR-carrying IONP less efficiently dephased the solution's water protons with concomitant high T2 and T1 readings, although the iron concentration was the same for all three samples. Additionally, these changes were cargo-dependent, as the high-content DiR preparation ([DiR]iONP⁼5-46 μM) facilitated higher T2 and T1 readings than the nanoparticles carrying lower amount of DiR ([DiR]iONP⁼1-57 μM). These results support the observation that loading cargo affected IONP's magnetic properties, regardless of the nanoparticles' type of polymeric coating and surface charge.

Feraheme® (ferumoxytol), a clinical IONP used for the treatment of chronic kidney disease, was tested as a surrogate drug delivery platform for cargo-dependent changes on magnetic properties. Thus, the fluorescent Taxol® derivative ([Flutaxl]Ferahemeg⁼30 μM), doxorubicin ([Doxorubicin]Feraheme®=828 μM) and DiR ([DiR]Feraheme®⁼920 μM) was loaded onto Feraheme®, which showed cargo-modulated alterations in the T2 and T1 signal (FIGS. 1C-D). These changes were also reflected in the resulting formulations relaxivities, where the r₂ and ri decreased after addition of the cargo (FIGS. 1E-F). Cargo-induced changes in r₂ and r_{\} were also observed when other compounds were loaded into the nanoparticles ([Drug] Feraheme®⁼100 μM), as well as in a Feraheme® preparation that was co-loaded with the androgen receptor antagonist MDV3100 and the PI3K inhibitor BEZ235 ([MDV3100] Feraheme®=250 μM and [BEZ235] Feraheme®=75 μM) (see infra Table 2 in Section IV under heading “Magnetic Properties of IONP Changes Upon Cargo Loading”). This selection of cargo included drugs that have discreet mechanisms of action and targets, which are located at the extracellular matrix and the cytoplasm, as well as at or within the plasma membrane. Since for these experiments a compact magnetic relaxometer was used, MRI was also used to image these changes in the nanoparticles' magnetic signal. Both unloaded suspensions of poly(acrylic acid) IONP and Feraheme® had low T2, while addition of payload gradually increased the T2, with respect to the molecular load of each sample (FIGS. 2A-B). Hence, it is contemplated that these changes in iron oxide nanoparticles' magnetic signal can be monitored with benchtop relaxometers and commonplace NMR analyzers, as well as animal and clinical MRI instruments.

TABLE 2
Relaxivities of unloaded (vehicle)
and drug-loaded Feraheme ® (Mean ± SE).

| | | Mechanism | [Drug] | Relaxivity | Relaxivity |
|---|---|---|---|---|---|
| Compound | MW | of action | (μm) | r₂(mM⁻¹s⁻¹) | r₁(mM⁻¹s⁻¹) |
| Vehicle | — | — | — | 109.5 ± 3.8 | 32.9 ± 1.4 |
| Alendronate | 325 | Bone | 100 | 104.1 ± 1.5 | 26.7 ± 0.6 |
| | | resorption | | | |
| | | inhibitor, | | | |
| | | farnesyl | | | |
| | | diphospate | | | |
| | | synthase | | | |
| | | inhibitor | | | |
| AZD8055 | 466 | mTOR | 100 | 48.8 ± 0.9 | 13.6 ± 0.4 |
| | | inhibitor | | | |
| | | (mTORC1, | | | |
| | | mTORC2) | | | |
| Dasatinib | 488 | Tyrosine | 100 | 80.4 ± 1.4 | 21.3 ± 0.8 |
| | | kinase | | | |
| | | inhibitor | | | |
| | | (BCR/ABL, | | | |
| | | Src) | | | |
| PU-H71 | 512 | Hsp90 | 100 | 88.4 ± 0.6 | 23.9 ± 0.5 |
| | | inhibitor | | | |
| GSI-34 | 534 | γ-secretase | 100 | 74.7 ± 1.3 | 20.5 ± 0.3 |
| | | inhibitor | | | |
| BKM120 | 580 | PI3K inhibitor | 100 | 98.1 ± 2.1 | 25.7 ± 0.7 |
| FR230 | 687 | Lipophilic | 100 | 37.9 ± 1.7 | 13.1 ± 0.9 |
| | | anti- | | | |
| | | angiogenic | | | |
| | | peptido- | | | |
| | | mimetic | | | |
| MDV3100 | 464 | Androgen | 250 | 87.9 ± 1.8 | 23.8 ± 0.3 |
| & BEZ | | receptor | 75 | | |
| 235 | 470 | antagonist | | | |
| | | PI3K inhibitor | | | |

The IONP's Molecular Payload Directly Affects The Accessibility Of Water Molecules. The inventors' contemplated that non-covalent intercalation of cargo molecules (payload) within the pockets of the polymeric coating of IONPs was preventing the interaction of water with the IONP's iron oxide core. Therefore, it was further contemplated that by obstructing the access and free diffusion of water molecules from the nanoparticles' outer relaxation sphere, the presence of cargo molecules was reducing the nanoparticles' capability to alter the bulk water's relaxation times. Thus, exemplary tests were done by incorporating increasing amounts of Flutaxl in the domains of Feraheme®'s coating, while monitoring the preparations' fluorescence emission and magnetic signal. Correlations were done on the amount of loaded drug with measured changes in the relaxation time T1 and T2, after dialyzing the nanoparticles to remove free non-intercalated drug. When the amount of Flutaxl added to the coated IONP was increased there were increases in both relaxation T1 and T2 times parallel to increasing fluorescence emission from the loaded Feraheme®, as opposed to unloaded nanoparticles that weren't fluorescent which had T1 of 402±7 ms and T2 of 121±2 ms (FIGS. 3A-C). The intercalation process had an apparent loading efficiency of approximately 80%, allowing large amounts of payloads to be entrapped within Feraheme®'s coating without affecting the nanoparticles' surface charge (FIGS. 12A-C). Therefore, it was contemplated that this method would find use for quantifying and monitoring the loading of non-fluorescent payloads into IONP. Furthermore, for excluding the possibility that the observed changes in the magnetic properties of IONP were caused by changes in the nanoparticles' size distribution instead of amounts of cargo loading, dynamic light scattering (DLS) analysis was performed. The data shown herein demonstrated that cargo-carrying IONPs had similar size distributions to that of corresponding IONPs without cargo, such that the average diameter was unaltered.

FIGS. 3D-G showing that the intercalation of cargo did not structurally change the nanoparticles.

### Incorporation Of Molecular Payload Within IONP's Coating Hindered The Efficient Diffusion Of Water Molecules Through The Nanoparticles' Coating.

As shown herein, higher T2 and T1 relaxation times were measured after IONP were loaded with cargo molecules, i.e. molecular payloads (FIG. 4A). Thus it was further contemplated that the presence of cargo within the pockets of the coating limited access of bulk water to the proximity of the iron oxide core. In order to measure whether bulk water was being excluded, a benchtop magnetic resonance reader (Minispec) was used to measure T2 changes of DiR-loaded poly(acrylic acid)-coated IONP between solutions with different concentrations of deuterium oxide (D2O). These measurements showed increased T2 after loading indicating the presence of cargo within the pockets of the coating limited access of bulk water to the proximity of the iron oxide core. Because the deuterium nucleus consists of a proton and a neutron, as opposed to hydrogen's single proton, deuterium has a different magnetic moment from hydrogen, allowing its use in the identification of the role of water and hydrogen protons, via nuclear magnetic resonance (NMR) spectroscopy.

While taking NMR measurements, it was discovered that as the concentration of D₂O increased, the T2 of solutions of DiR-loaded IONP decreased, showing that as D₂O became more abundant in the solution than H₂O, the presence of intercalated cargo did not exert the initial effect on the water proton relaxation times, due to the low abundance of water (FIG. 4B). This change in T2 was attributed to the capability of cargo to obstruct water molecules from the nanoparticles' vicinity, eventually causing T2 increases during cargo loading. Surprised by these findings, it was contemplated whether cargo incorporation within Feraheme® impairs the diffusion of water molecules within Feraheme®'s coating, explaining the observed increases in T1. Thus diffusion-weighted imaging and MM was used to obtain results emanating from the apparent diffusion coefficient map. It was found that the Flutaxl- and doxorubicin-loaded Feraheme® had lower diffusion coefficients than the unloaded Feraheme® (FIGS. 4C-D), showing that cargo intercalation (loading) obstructed the free diffusion of water molecules.

T2 Values Decreased and T1 Values Increased with Particle Aggregation.

Target-induced clustering of IONP was used in sensitive assays for the detection of numerous biomolecules and targets (Grimm, et al., Novel nanosensors for rapid analysis of telomerase activity. Cancer Res 2004, 64(2):639-43, Perez, et al., Integrated nanosensors to determine levels and functional activity of human telomerase. Neoplasia 2008, 10 (10): 1066-72; Perez, et al., Magnetic relaxation switches capable of sensing molecular interactions. Nat Biotechnol 2002, 20 (8):816-20, each of which is herein incorporated by reference in its entirety). Specifically, it was demonstrated that the nanoparticles form extensive supramolecular assemblies in the presence of their target (Perez, et al., Magnetic relaxation Switches Capable of Sensing Molecular Interactions. Nat Biotechnol 2002, 20 (8), 816-20; Koh, et al., Nanoparticle-target Interactions Parallel Antibody-protein Interactions. Anal Chem 2009, 81 (9), 3618-22; Kaittanis, et al., The Assembly State between Magnetic Nanosensors and Their Targets Orchestrates Their Magnetic Relaxation Response. J Am Chem Soc 2011, 133 (10), 3668-76, each of which is herein incorporated by reference in its entirety). The formation of these assemblies that consisted of multiple nanoparticles was predominantly associated with T2 decreases and no reported effect on T1.

Since this result was different from the observed increases in T2 and T1 during cargo incorporation, T1, was measured for affects during Feraheme®'s aggregation. As a model target-induced clustering system, Concanavalin A (Con A), a protein that has high affinity towards carbohydrates, (Asian, et al., Nanogold-plasmon-resonance-based Glucose Sensing. Anal Biochem 2004, 330 (1), 145-55; Yoshizumi, et al., Self-assembled Monolayer of Sugar-carrying Polymer Chain: Sugar Balls from 2-methacryloyloxyethyl D-glucopyranoside. Langmuir 1999, 15 (2), 482-488, each of which is herein incorporated by reference in its entirety) was used to facilitate the clustering of Feraheme®, since it was coated with carboxymethyl dextran. Addition of the Con A ([Con A]_{fma}f=50 μg/mL) to Feraheme® ([Fe]=23 g/mL) induced decrease in the solution's T2 but increase in the TI (FIGS. 5A-B), with nanoparticle aggregation confirmed with DLS (FIG. 5C). Likewise, the differential effect on IONP's magnetic properties was reflected on their relaxivities, with the r₂ increasing and r_{\} decreasing since they are inversely proportional to the relaxation times T2 and T1 respectively (FIGS. 13A-B). Moreover, when enhanced nanoparticle aggregation was facilitated in the presence of excess dextran ([Dextran]=2.5 mg/mL), marked increase in the T1 value was observed, with the T2 decreasing (FIGS. 5A-C) due to Feraheme®'s Con A-induced clustering. These results demonstrated that IONP aggregation mediated significant decreases in the spin-spin relaxation times (T2), while promoting increases on T1, which become prominent as the aggregates become bigger. This may explain why previous studies did not identify the effect of nanoparticle clustering on the T1 process (Taktak, et al., Multiparameter Magnetic Relaxation Switch Assays. Anal Chem 2007, 79 (23), 8863-9, herein incorporated by reference in its entirety) as one contribution is the extent of the aggregation state. In summary, these results showed that cargo incorporation and the associated T2 and T1 increases were discerned from nanoparticle clustering and its effect on IONP magnetic properties, thus showing that sensitive characterization of diverse cargo-loaded formulations would be of use in research and clinical settings.

pH-Dependent Release of Cargo Alters the Magnetic Properties of Loaded IONP.

One application for intercalated drugs in coated !ONPs is the use of IONP as a drug delivery platform, which allows for control of loading (and release) by changes in T2 and T1. Ideally, the cargo should be retained at physiological conditions and released in the presence of environmental cues, such as abnormally low pH. This feature is ideal for cancers that exhibit acidic interstitial pH, due to upregulated glycolysis as a result of signaling and metabolic alterations, collectively described as the Warburg effect (Vander Heiden,et al., Understanding the Warburg effect: the metabolic requirements of cell proliferation. Science 2009, 324 (5930), 1029-33, herein incorporated by reference in its entirety).

In order to examine the potential use of Feraheme® as a smart drug delivery system, the stability of drug-loaded Feraheme® in phosphate-buffered saline (PBS) at physiologic pH of 7.4 was investigated. Incubation of doxorubicin-loaded Feraheme® in pH-adjusted buffer for 24 hours did not reveal major changes in the T2, and T1, spanning the pH range encountered during physiological conditions (FIGS. 15A-B). Since doxorubicin is fluorescent and was used in spectroscopic and fluorescence microscopy studies (for example, see, Santra, et al., Cell-specific, Activatable, and Theranostic Prodrug for Dual-targeted Cancer Imaging and Therapy. J Am Chem Soc 2011, 133 (41): 16680-8), herein incorporated by reference in its entirety), the nanoparticle's fluorescence emission was monitored across the pH range shown in FIG. 15C.

However there were significant changes in fluorescence emission intensity. Changes in T2 and T1 were observed at a lower pH of 7.0 thus further experiments were done to identify whether the cargo was being released upon acidification of the aquatic milieu. Employing a dialysis chamber to separate the nanoparticles from the potentially released drug, doxorubicin-carrying Feraheme® was incubated in IX PBS adjusted to pH 6.8 and 6.0. Rapid decreases in T2 and T1 were observed under these mildly acidic conditions (FIGS. 6A-B), which were accompanied with decreases in the nanoparticles' fluorescence emission (FIGS. 6C) due to release of doxorubicin as confirmed with HPLC-based spectrophotometry (FIGS. 6D). On the other hand, no changes in T2 and T1 were observed at pH 7.2, likely due to stable accommodation of doxorubicin within Feraheme®'s coating, which is supported by the preparation's unaltered fluorescence emission. To further confirm that these changes were mediated by cargo release, DLS was performed prior and after incubation at these pH levels. Results indicated that the nanoparticle size and distribution were constant throughout the experiment, with the nanoparticles being stable after 2 h at pH 6.0 (FIG. 6E-F). Plausibly, release of cargo at acidic pH may have been facilitated due to disturbance of the weak electrostatic interactions that mediate the association of the drug with the nanoparticles' polymeric backbone secondary to hydrophobic interactions. It is possible that changes in the protonation of the polymer's side chains may disturb hydrogen bonding and van der Waals interaction between the IONP's coating and the cargo, thus triggering release at lower pH. This further demonstrates that the effect of cargo intercalation within the shell of a coated IONP of the present inventions was reversible, since payload release facilitated recovery of their magnetic properties, that lead to decreased T2 and T1.

Therefore, Feraheme® is contemplated for use for delivery of chemotherapeutic cargo to the tumor, with the lesion's acidic pH serving as an endogenous trigger for rapid drug release at the tumor's vicinity, maximizing therapeutic efficacy.

### Effective Feraheme®-Based Delivery of Chemo Therapeutics.

After establishing that cargo release caused decreased T2 and T1, cargo-loaded IONPs were used in testing whether IONP would deliver therapeutic payloads within cancer cells. Serum stability measurements were taken for establishing stability of cargo loaded coated IONPs in vivo. Serum alone had a T2 value of 600±10 ms and T1 of 1700±30 ms, which remained unaltered during the course of the study. DiR-carrying Feraheme® was found to be stable for up to 8 days in sterile fetal bovine serum (FIGS. 15A-D), with no changes in the relaxation times. However, doxorubicin- and Flutaxl-loaded nanoparticles released their payload having different types kinetic profiles, as indicated by concomitant changes in the relaxation times (FIGS. 7A-D). Specifically, doxorubicin was completely released from it's IONP carrier within less than four days, which resulted in relaxation times that were comparable to those of the unloaded nanoparticles whose T2 and T1 remained constant throughout the study (FIGS. 7A-B). On the other hand, the Taxol® derivative Flutaxl (a fluorescent taxol derivative) was used that demonstrated sustained release in serum over the ten-day duration of the study, where approximately 50% of the drug was released after 7 days (FIGS. 7C-F). These different release patterns were contemplated to be the result of different physical and chemical characteristics of each payload molecule. For example, doxorubicin, a small anthracycline DNA intercalator (MW: 580), might be released faster, due to lower hydrogen bonding formation with the shell polymer that resulted an overall weaker association with the nanoparticles. On the other hand, Flutaxl, a fluorescent microtubule-stabilizing diterpene (MW: 1337), has multiple oxygen and nitrogen atoms would facilitate a stronger noncovalant association within the nanoparticle's polymeric coating molecule.

Additionally, Flutaxl has multiple molecular structures (segments) that would favor multiple hydrophobic interactions with shell molecules. In fact, this latter observation is contemplated to contribute to the stabilization of DiR (MW: 1013) within the nanoparticle shells due to the presence of two eighteen-carbon-long aliphatic chains.

Further, the inventors' contemplated that IONPs having multiple types of molecular payloads would be able to target several oncogenic pathways instead of merely having one drug for one targeted interaction. Such a strategy was contemplated to increase the drugs' release at the tumor site while maximizing their circulation time, in a process that is orchestrated by the payload's intrinsic characteristics and initiated by the tumor's aberrant glycolytic activity, without subjecting the drugs to modification.

Furthermore, since enhanced permeability and retention (EPR) was a feature of tumors, it was determined whether cancer cells, such as the prostate cancer cell LNCaP, would uptake the cargo-loaded Feraheme® thus facilitating the intracellular release of the cargo from the nanoparticles. Incubation of LNCaP with the Doxorubicin-loaded Feraheme® for 48 h resulted in significant nanoparticle uptake, as indicated by the enhanced fluorescence due to the presence of doxorubicin, which was determined through fluorescence microscopy (FIG. 8A). In addition to determining whether LNCaP cells would uptake doxorubicin-loaded Feraheme®, further studies investigated whether prostate cancer cells would uptake Feraheme® that carried larger cargo (i.e. larger molecules), such as the near-infrared fluorophore DiR. After 48 h incubation with the loaded nanoparticles, the cells were washed, trypsinized and the resulting cell pellets were resuspended in IX PBS pH 7.4. Quantification of the cell-associated DiR fluorescence was achieved through the Odyssey imager, which demonstrated high fluorescence emission at 800 nm excitation in cells treated with the DiR-carrying Feraheme® (FIGS. 8B).

Additionally, experimental results obtained during the development of the present inventions showed that in addition to nanoparticle uptake, the unloading of DiR within the cells partially restored Feraheme®'s magnetic properties, which approached those of the original pre-loaded state. The r₂ and r_{\} relaxivities of the unloading nanoparticles (where intercalated cargo molecules were disassociating from noncovlant bonds with coating molecules) were higher than those of the loaded Feraheme® (FIGS. 8C-D). Taken together, these findings show that the effects of the cargo on Feraheme®'s magnetic properties were reversible, since payload intercalation relied on non-covalent bonding between shell molecules and loaded molecules. Thus, this may facilitate the monitoring of drug loading on Feraheme® and release via magnetic resonance methods, for in vitro and in vivo applications. Particularly for drugs with poor aqueous solubility, this approach can significantly improve their circulation time and stability with a clinically approved nanoparticle-based delivery system. Since the intercalation process was reversible and does not modify the drug chemically, these drugs retain their structure—function mode of action, exerting their biological effect once released at the acidic tumoric bed or within cancer cells, upon uptake through endocytic processes. Findings during the development of the present inventions described herein, demonstrated that magnetic relaxation can be used as an attractive method for the sensitive characterization of non-fluorescent payloads carried by (super)paramagnetic nanoparticles in pharmacological and clinical studies, utilizing portable relaxometers, (Taktak, et al., Multiparameter Magnetic Relaxation Switch Assays. Anal Chem. (2007) 79(23):8863-9; Issadore, et al, Miniature Magnetic Resonance System for Point-of-care Diagnostics. Lab Chip (2011) 11(13):2282-7, each of which is herein incorporated by reference in its entirety). NMR analyzers and MRI and expanding the applications of IONP in new areas, such as chemoprevention and tumor-directed, personalized therapy, such as cargo-loaded iron oxide nanoparticles were tested on cancer cells in order to measure the release of their cargo intracellularly. Control studies demonstrated absence of cytotoxicity in cells treated with unloaded Feraheme®, in accordance with other iron oxide nanoparticle formulations that have different polymeric coatings than this clinically approved agent. Therefore, the in vitro toxicity of Feraheme® carrying chemotherapeutic agents was investigated. Surprisingly, treatment of human prostate adenocarcinoma cells (LNCaP) with drug-carrying Feraheme® resulted in significant cell death, which was more pronounced than treatment with equimolar concentration of the free drug (FIG. 9A). For instance, doxorubicin-loaded Feraheme® induced more than 75% reduction in cell viability, as opposed to doxorubicin alone that caused 60% decrease. Similarly, Flutaxl -carrying nanoparticles were 10% more cytotoxic than the nascent drug, further supporting the notion that delivery of chemotherapeutics with this nanoplatform does not compromise the drug's cytotoxic activity, due to the absence of covalent modifications during the intercalation process. Apart from conventional chemotherapeutics that target DNA synthesis and mitosis, anticancer drugs that target enzymes involved in signal transduction and cholesterol/sterol biosynthesis were tested. These results indicated that the loaded-Feraheme® preparations were more effective than free Alendronate and BKM120 (FIG. 9B), likely due to the improved aqueous stability and bioavailability that the nanoparticles confer to their cargo. Effective Treatment With Duel Loaded IONP.

Because certain cancers, such as prostate cancer, have more than one pathway involved in oncogenesis and metastasis, Feraheme® was utilized as a drug delivery vehicle for combinatorial therapy. Specifically, since in prostate cancer the crosstalk between the androgen receptor pathway and the PI3 cascade leads to resistance to agents targeting one of the two pathways, (Carver, et al., Reciprocal feedback regulation of PI3K and androgen receptor signaling in PTEN-deficient prostate cancer. Cancer Cell (2011), 19 (5), 575-86), herein incorporated by reference in its entirety), developing a strategy that targeted both pathways would be ideal, leading to improved therapeutic efficacy.

Thus, BEZ235, a PI3K inhibitor, and the androgen receptor antagonist MDV3100 were intercalated together into the outer coating of the same IONP, in order to deliver both drugs within prostate cancer cells. The prostate cancer cell line LNCaP was treated with this duel loaded IONP. A prostate cancer cell line LNCaP, which has a functional AR cascade, showed that Feraheme® carrying both BEZ235 and MDV3100 led to more than 30% reduction in cell viability, as opposed to cells treated with one free drug or a combination of both free drugs (FIG. 9C).

In vivo studies revealed that doxorubicin-carrying Feraheme® is more effective than free doxorubicin, after three iv injections of either free or intercalated doxorubicin (FIG. 9D). Specifically, the mice were treated with equal concentrations of the drug on day 0, 2 and 6, and tumor growth was determined with microcalipers. Control animals were treated with 100 μ\hat{ι}, of 10% DMSO-containing PBS to match the DMSO content of the free doxorubicin injection, since the drug was dissolved in DMSO and diluted to the desired concentration in PBS. On day 6 and 8, the tumors were measured, and the change in tumor growth was determined by comparing the volume of tumors from treated animals with those of control ones. Surprisingly, doxorubicin-loaded Feraheme® IONPs were able to achieve 10% tumor regression after two injections, as opposed to some tumor growth after treatment with free doxorubicin. In fact, after three injections, when the tumors were measured on day 8 for each of these treatments, the drug-loaded Feraheme® was able to suppress tumor growth by almost 50% as opposed to 34% suppression from the use of free drug. These measurements showed that treatment with an ampipathic agent that was intercalated within Feraheme® enhanced its bioavailability. This enhancement was contemplated to be the result of the loaded coated Feraheme®'s capability to specifically deliver drug within tumors at effective dosages while preventing association of drug with proteins and lipids in solution. Whereas nonspecific association of free drug with proteins and lipids in solution reduced the effective dosage, i.e. the amount of administered drug compared with effect.

### EXPERIMENTAL

The following examples serve to illustrate certain embodiments and aspects of the present invention and are not to be construed as limiting the scope thereof. In the experimental disclosures which follow, the following abbreviations apply: N (normal); M (molar); mM (millimolar); μM (micromolar); mol (moles); mmol (millimoles); μηιo\hat{ι} (micromoles); nmol (nanomoles); pmol (picomoles); g (grams); mg (milligrams); μg (micrograms); ng (nanograms); pg (picograms); L and (liters); ml (milliliters); μ\hat{ι}

(microliters); cm (centimeters); mm (millimeters); μπι (micrometers); nm (nanometers); U (units); min (minute); s and sec (second); deg (degree); pen (penicillin), and ° C. (degrees Centigrade/Celsius).

### EXAMPLE L

The following are exemplary materials and methods used during the development of the present inventions.

### A. Materials.

Chemicals were of analytical grade unless otherwise stated herein. Ferrous and ferric chloride salts (FeCl2.4H2O and FeCl3.6¾0) were obtained from Fluka while deuterium oxide (D2O) was from Acros (Pittsburgh, Pa., United States).

Poly(acrylic acid) (PAA, MW 1.8 kDa), ammonium hydroxide, hydrochloric acid and dimethyl sulfoxide (DMSO) were obtained from Sigma-Aldrich. Dextran (10 kDa) was acquired from Pharmacosmos (Holbaek Denmark), while the carbohydrate-binding lectin Concanavalin A (Con A) was purchased from Sigma-Aldrich. Payload included but was not limited to the following compounds: Alendronate (MW: 325) from Sigma Aldrich, AZD8055 (MW: 466) from Selleck Chemicals, BEZ235 (MW: 470) from Cayman Chemicals, BKM120 (MW: 580) was a generous gift from Professor Lewis Cantley (Harvard Medical School, Beth Israel Deaconess Medical Center), Dasatinib (MW: 488) from Selleck Chemicals, DiR (l,r-dioctadecyl-3,3,3′,3′-tetramethylindo-tricarbocyanine iodide, MW: 1013) from Invitrogen, doxorubicin (Adriamycin, MW: 580) from Selleck Chemicals, Flutaxl—a fluorescent Taxol® derivative (MW: 1337) from Tocris Bioscience, FR230 (MW: 687) was kindly provided by Dr. Horst Kessler (Technische Universitat Munchen), GSI-34 (MW: 534) was provided by Dr. Yueming Li (MSKCC), MDV3100 (MW: 464) was generously provided by Professor Charles Sawyers (MSKCC) and PU-H71 (MW: 512) was provided by Dr. Gabriela Chiosis (MSKCC) . Stocks of these chemicals were prepared in DMSO, and stored at −20° C. until further use.

Commercially available iron oxide nanoparticle preparations such as NH2-nanomag®-D-spio and Feraheme® were obtained from Micromod Partikeltechnologie GmbH (Rostock, Germany; NH2-nanomag®-D-spio) and AMAG Pharmaceuticals (Lexington, Mass.; Feraheme®).

### B. Preparation of Iron Oxide Nanoparticles.

Poly(acrylic acid)-coated iron oxide nanoparticles were prepared with the alkaline precipitation method, involving the rapid mixing of ferrous and ferric chloride in ammonium hydroxide that was followed by addition of the polymer solution (for example, see, Santra, et al., Drug/dye-loaded, multifunctional iron oxide nanoparticles for combined targeted cancer therapy and dual optical/magnetic resonance imaging. Small, 5(16):1862-8 (2009), herein incorporated by reference in its entirety). To remove excess reagents and byproducts, the nanoparticles were washed, concentrated and reconstituted in pH 7.4 phosphate buffered saline (PBS), with a KrosFlo Research II TFF system that was equipped with a 10 kDa column (Spectrum). The nanoparticles were stored at 4° C. until further use, without precipitation being observed, similar to the aminated nanoparticles obtained from Micromod that were used without additional preparation. Feraheme® was subjected to magnetic separation using a IX PBS -equilibrated LS25 MACS column (Miltenyi), in order to isolate IONP with good magnetic properties from free polymer in the solution. Subsequently, Feraheme® was stored at 4° C. until further use.

C. Drug loading into Iron Oxide Nanoparticles.

Intercalation of the molecular payload was achieved using a modified solvent-diffusion-based protocol, facilitating the entrapment of hydrophobic molecules within IONP's polymeric coating. In general, the nanoparticles (25 μ\hat{ι}, of either PAA-IONP or NH2-nanomag®-D-spio, 30 μ\acute{ι}, of Feraheme®) were resuspended in 500 μL-distilled water, whereas the cargo was diluted to the desired concentration in DMSO (final volume of payload solution was 100 μ\hat{ι}). The fluorophore or drug payload solution was added dropwise to the nanoparticle solution under vortexing (1000 rpm) at room temperature, without visible precipitation. Subsequently, the preparation was subjected to dialysis in a small-volume dialysis chamber (MWCO 3000, Fisher) against IX PBS. The cargo-carrying IONP were subsequently stored in the dark at 4° C., until further use.

### D. Nanoparticle Characterization.

Size of IONPs were determined through dynamic light scattering (DLS) (Nano-ZS, Malvern, Westborough, Mass. (MA), United States). The same instrument was used in nanoparticle surface charge measurement potential), whereas to determine Feraheme®'s nanoparticle concentration the NS500 instrument was utilized (NanoSight, Duxbury, Mass.). Magnetic relaxation measurements, including r and r₂ relaxivities, were determined with a 0.47 T mq20 NMR analyzer (Minispec, Braker, Billerica, Mass.). For T2 measurements a CPMG pulse-echo train with a 1.5 ms interpulse spacing was used, whereas the T1 sequence varied the interpulse spacing from 5 ms up to 8500 ms. The preparations' iron concentration was determined spectrophotometrically as previously reported (Nath, et al., Synthesis, magnetic characterization and sensing applications of novel dextran-coated iron oxide nanorods. Chem Mater, 21 (8): 1761-1767 (2009), herein incorporated by reference in its entirety) using a SpectraMax M5 instrument from Molecular Devices. Fluorescence emission measurements were performed using the SpectraMax M5, as well as an Odyssey near-infrared imaging station (LI-COR Biosciences), equipped with two solid-state lasers for excitation at 685 and 785 nm. To determine the cargo load of each preparation, the following molar extinction coefficients were used: ε Doxorubicin=11500 M″¹ cm″¹ at 480 nm, ε Flutaxl=52000 M″¹ cm″¹ at 495 nm and 8DiR=270000 M″¹ cm″¹ at 748 nm. Stability experiments were performed in pH-adjusted phosphate buffered saline, whereas serum experiments were performed at 37° C., using fetal bovine serum obtained from Gemini Bio-products. Release of doxorubicin from drug-loaded Feraheme® was performed using a dynamic dialysis setup, as previously described (Santra, et al., Aliphatic hyperbranched polyester: a new building block in the construction of multifunctional nanoparticles and nanocomposites. Langmuir, 26(8):5364-73 (2010), herein incorporated by reference in its entirety). A dialysis chamber was utilized (MWCO 3000, Fisher), containing doxorubicin-loaded Feraheme® in either pH 7.2 or pH 6.8 IX PBS. The nanoparticles were dialyzed against the corresponding pH-adjusted buffer at room temperature and under constant stirring (150 rpm), where at regular time intervals aliquots from the external aqueous milieu of the device were collected for further analysis. The collected samples were analyzed via a Beckman Coulter HPLC instrument, equipped with a CI 8 reverse phase column and set to monitor doxorubicin's absorbance at 480 nm mixing An animal MM from Bruker Biospin operating at 4.7T and a 35-mm radiofrequency coil were used to image phantoms of the nanoparticle preparations that were spotted on a microplate.

In vitro drug release from loaded IONP. LNCaP cells were grown to confluence, on a 12-well poly(lysine)-coated plate in 10% FBS-containing RPMI medium at 37° C., 5% CO₂. The medium was aspirated, and the cells were supplemented with 1 mL fresh media, plus 50 μ\hat{ι}. of either empty (vehicle), Doxorubicin-loaded Feraheme® or DiR-loaded Feraheme®. After 48 h, the cells treated with Doxorubicin-loaded Feraheme® were examined under a Nikon Eclipse T1 fluorescence microscope, in order to determine the nanoparticle uptake. Likewise, following 48h-long incubation at 37° C., 5% CO₂, the cells treated with vehicle and DiR-loaded nanoparticles were trypsinized and subjected to centrifugation at 1000 rpm for 6 min. The resulting pellets were then resuspended in 400 μ\hat{ι}, IX PBS and aliquoted in two eppendorf tubes for fluorescence emission and magnetic relaxation measurements, using the near-infrared imager (LI-COR) and the benchtop relaxometer (Bruker). For near-infrared fluorescence, excitation was achieved at 785 nm, with emission recorded at 800 run; with the instrument settings set as follows: focus offset=4 mm, intensity=0.5 and resolution=169 μπι. The iron content of the cell pellets was determined as described above, with untreated samples of equal cell numbers serving as control.

Cell viability and in vivo studies. LNCaP cells were seeded on black-walled, clear bottom 96-well plates at a cell density of 10,000 cells per well, supplemented with 100 10% FBS-containing RPMI medium. After 24 h growth at 37° C., 5% CO₂, the cells were treated with 10 μ\hat{ι}, per well of either free or intercalated drug ([Doxorubicin]_{fmal}=7.5 μM, [Flutaxl] [symbol] μM, [symbol] μM, [MDV3100]_{fina}i=5.6 μM), followed by 48 h incubation (37° C., 5% CO₂). Controls included cells incubated with unloaded nanoparticles or DMSO, corresponding to the free drug's final solvent concentration. Subsequently, the old medium was aspirated, and cell viability was assessed via the Alamar Blue method (Invitrogen). Briefly, the cells were supplemented with 10%-alamar-blue-containing medium (10% FBS-containing RPMI), followed by 3 h incubation in a humidified incubator (37° C., 5% CO₂) and recording of fluorescence emission [symbol] ran, λ_{ειλ}=585 nm) with the SpectraMax M5 plate reader. Nude, male mice (n=10) bearing PC3 tumors on their flanks were treated on day 0, day 2 and day 6 with 100\L either doxorubicin alone or doxorubicin-loaded Feraheme®, both at a final doxorubicin concentration of 0.28 mM. Changes in tumor size were evaluated with a microcaliper, and at the end of the 8-day study the mice were euthanized, according to the Institutional Animal Care and Use Committee guidelines.

### Data Analysis.

Experiments were performed in triplicate unless otherwise stated, with the results are presented as mean±SEM. The data were analyzed in Prism (GraphPad Software), whereas the MR images were processed through the OsiriX DICOM viewer.

### EXAMPLE II

Exemplary Demonstration of How Cargo Incorporation Affects the Magnetic Properties of IONP.

Incorporation of molecular payload on iron oxide nanoparticles was contemplated to have facilitated increases in solution's T2 and T1 relaxation times, likely due to displacement of water molecules from IONP's outer relaxation sphere. In order to test whether intercalation of cargo onto coated IONPs, poly(acrylic acid)-coated IONPs and a variety of different molecular weight cargos were tested, including fluorophores (Di) and chemotherapeutics (Doxorubicin and Flutaxl ; a fluorescent Taxol® derivative). After cargo intercalation within the pockets of the nanoparticles' polymeric coating via the solvent diffusion method, distinct changes were observed in all cargo-carrying preparations, as opposed to the control unloaded (vehicle) IONP preparation (FIGS. 1A-B). Specifically, the higher the concentration of the payload within the nanoparticles, the more pronounced the changes in the relaxation times were observed. For instance, a preparation of IONP with high Flutaxl levels ([Flutaxl]IONP=30 μM) less efficiently influenced the solution's proton relaxation times than a corresponding preparation with a lower Flutaxl load ([Flutaxl [symbol] μM). Since T2 and T1 are inversely proportional to a contrast agent's spin-spin (r₂) and spin-lattice (r) relaxivities, the high-load Flutaxl-carrying IONP had r₂ and r_{\} relaxivities lower than those of the low-load Flutaxl preparation, indicating that the observed changes are mediated by the amount of cargo within the nanoparticle (FIGS. 10A-B). In line with these findings, similar results were obtained when low and high DiR-carrying IONP formulations were tested, [symbol] μM, respectively), where the high-load formulation resulting in higher relaxation times (FIGS. 1A-B, FIGS. 10A-B).

Furthermore, intercalation of doxorubicin increased both the T2 and T1 signal, indicating that nascent chemotherapeutics can be loaded within the nanoparticles and induce changes in the preparation's magnetic properties ([Doxorabicin]ioNP=12.5 μM) (FIGS. 1A-B, FIGS. 10A-B). Hence, these results suggest that this phenomenon is modulated by cargo incorporation within the IONP's poly(acrylic acid) coating, leading to IONP with compromised magnetic properties.

### EXAMPLE III

Exemplary Demonstration of Magnetic Properties of IONP That Changed Upon Cargo Loading.

The inventors subsequently examined whether the change in magnetic properties was observed in other IONP, which were stabilized with other polymeric coatings. Indeed, aminated IONP behaved similarly to their negatively charged poly(acrylic acid) counterparts, in the presence of molecular cargo (FIGS. 11A-B). Compared to the corresponding vehicle preparation, the aminated DiR-carrying IONP less efficiently dephased the solution's water protons with concomitant high T2 and T1 readings, although the iron concentration was the same for all three samples. Additionally, these changes were cargo-dependent, as the high-content DiR preparation ([DiR]iONP⁼5.46 μM) facilitated higher T2 and T1 readings than the nanoparticles carrying lower amount of DiR ([DiR]iONP⁼1-57 μM). Taken together, these results support that loading cargo affects IONP's magnetic properties, regardless of the nanoparticles' polymeric coating and surface charge.

Feraheme® (ferumoxytol), a clinical IONP used for the treatment of chronic kidney disease, was tested as a surrogate drug delivery platform for cargo-dependent changes on magnetic properties. Thus, the fluorescent Taxol® derivative ([Flutaxl]Ferahemeg⁼30 μM), doxorubicin ([Doxorabicin]Feraheme®⁼828 μM) and DiR ([DiR]Feraheme®⁼⁼920 μM) was loaded onto Feraheme®, which showed cargo-modulated alterations in the T2 and T1 signal (FIGS. 1C-D). These changes were also reflected in the resulting formulations relaxivities, where the r₂ and r_{\} decreased after addition of the cargo (FIGS. 1E-F). Cargo-induced changes in r % and r_{\} were also observed when other compounds were loaded into the nanoparticles ([DrugjFeraheme®\hat{ }¹\hat{ }\hat{ } uM), as well as in a Feraheme® preparation that was co-loaded with the androgen receptor antagonist MDV3100 and the PI3K inhibitor BEZ235 ([MDV3100]Ferahemeg=250 μM and [BEZ235]Feraheme®=75 μM) (Table 3). This selection of cargo included drugs that have discreet mechanisms of action and targets, which are located at the extracellular matrix and the cytoplasm, as well as at or within the plasma membrane. Since for these experiments a compact magnetic relaxometer was used, MRI was also used to image these changes in the nanoparticles' magnetic signal. Both unloaded suspensions of poly(acrylic acid) IONP and Feraheme® had low T2, while addition of payload gradually increased the T2, with respect to the molecular load of each sample (FIGS. 2A-B). Hence, it is contemplated that these changes in iron oxide nanoparticles' magnetic signal can be monitored with benchtop relaxometers and commonplace NMR analyzers, as well as animal and clinical MRI instruments.

TABLE 3
Relaxivities of unloaded (vehicle)
and drug-loaded Feraheme ® (Mean ± SE).

| | | Mechanism | [Drug] | Relaxivity | Relaxivity |
|---|---|---|---|---|---|
| Compound | MW | of action | (μm) | r₂(mM⁻¹s⁻¹) | r₁(mM⁻¹s⁻¹) |
| Vehicle | — | — | — | 109.5 ± 3.8 | 32.9 ± 1.4 |
| Alendronate | 325 | Bone | 100 | 104.1 ± 1.5 | 26.7 ± 0.6 |
| | | resorption | | | |
| | | inhibitor, | | | |
| | | farnesyl | | | |
| | | diphospate | | | |
| | | synthase | | | |
| | | inhibitor | | | |
| AZD8055 | 466 | mTOR | 100 | 48.8 ± 0.9 | 13.6 ± 0.4 |
| | | inhibitor | | | |
| | | (mTORC1, | | | |
| | | mTORC2) | | | |
| Dasatinib | 488 | Tyrosine | 100 | 80.4 ± 1.4 | 21.3 ± 0.8 |
| | | kinase | | | |
| | | inhibitor | | | |
| | | (BCR/ABL, | | | |
| | | Src) | | | |
| PU-H71 | 512 | Hsp90 | 100 | 88.4 ± 0.6 | 23.9 ± 0.5 |
| | | inhibitor | | | |
| GSI-34 | 534 | γ-secretase | 100 | 74.7 ± 1.3 | 20.5 ± 0.3 |
| | | inhibitor | | | |
| BKM120 | 580 | PI3K inhibitor | 100 | 98.1 ± 2.1 | 25.7 ± 0.7 |
| FR230 | 687 | Lipophilic | 100 | 37.9 ± 1.7 | 13.1 ± 0.9 |
| | | anti- | | | |
| | | angiogenic | | | |
| | | peptid-o | | | |
| | | mimetic | | | |
| MDV3100 | 464 | Androgen | 250 | 87.9 ± 1.8 | 23.8 ± 0.3 |
| & BEZ 235 | 470 | receptor | 75 | | |
| | | antagonist | | | |
| | | PI3K inhibitor | | | |

### EXAMPLE IV

Exemplary Demonstration of IONP's Molecular Payload That Directly Affected the Accessibility of Water Molecules.

The inventors' contemplated that non-covalent intercalation of cargo molecules (payload) within the pockets of the polymeric coating of IONPs was preventing the 5 interaction of water with the IONP's iron oxide core. Therefore, it was further contemplated that by obstructing the access and free diffusion of water molecules from the nanoparticles' outer relaxation sphere, the presence of cargo molecules was reducing the nanoparticles' capability to alter the bulk water's relaxation times. Thus, exemplary tests were done by incorporating increasing amounts of Fluta 1 in the domains of 0 Feraheme®'s coating, while monitoring the preparations' fluorescence emission and magnetic signal. Correlations were done on the amount of loaded drug with measured changes in the relaxation time T1 and T2, after dialyzing the nanoparticles to remove free non-intercalated drug. When the amount of Flutaxl added to the coated IONP was increased there were increases in both relaxation T1 and T2 times parallel to increasing fluorescence emission from the loaded Feraheme®, as opposed to unloaded nanoparticles that weren't fluorescent which had T1 of 402±7 ms and T2 of 121±2 ms (FIGS. 3A-C). The intercalation process had an apparent loading efficiency of approximately 80%, allowing large amounts of payloads to be entrapped within Feraheme®'s coating without affecting the nanoparticles' surface charge (FIGS. 12A-C).

Therefore, it was contemplated that this method would find use for quantifying and monitoring the loading of non-fluorescent payloads into IONP. Furthermore, for excluding the possibility that the observed changes in the magnetic properties of IONP were caused by changes in the nanoparticles' size distribution instead of amounts of cargo loading, dynamic light scattering (DLS) analysis was performed. The data shown herein demonstrated that cargo-carrying IONPs had similar size distributions to that of corresponding IONPs without cargo, such that the average diameter was unaltered. FIGS. 3D-G showing that the intercalation of cargo did not structurally change the nanoparticles.

### EXAMPLE V

Exemplary Demonstration of Incorporation Of Molecular Payload Within IONP's Coating That Hindered The Efficient Diffusion Of Water Molecules Through The Nanoparticles' Coating.

As shown herein, higher T2 and T1 relaxation times were measured after IONP were loaded with cargo molecules, i.e. molecular payloads (FIG. 4A). Thus it was further contemplated that the presence of cargo within the pockets of the coating limited access of bulk water to the proximity of the iron oxide core. In order to measure whether bulk water was being excluded, a benchtop magnetic resonance reader (Minispec) was used to measure T2 changes of DiR-loaded poly(acrylic acid)-coated IONP between solutions with different concentrations of deuterium oxide (D2O). These measurements showed increased T2 after loading indicating the presence of cargo within the pockets of the coating limited access of bulk water to the proximity of the iron oxide core. Because the deuterium nucleus consists of a proton and a neutron, as opposed to hydrogen's single proton, deuterium has a different magnetic moment from hydrogen, allowing its use in the identification of the role of water and hydrogen protons, via nuclear magnetic resonance (NMR) spectroscopy.

While taking NMR measurements, it was discovered that as the concentration of D2O increased, the T2 of solutions of DiR-loaded IONP decreased, showing that as D2O became more abundant in the solution than H2O, the presence of intercalated cargo did not exert the initial effect on the water proton relaxation times, due to the low abundance of water (FIG. 4B). This change in T2 was attributed to the capability of cargo to obstruct water molecules from the nanoparticles' vicinity, eventually causing T2 increases during cargo loading. Surprised by these findings, it was contemplated whether cargo incorporation within Feraheme® impairs the diffusion of water molecules within Feraheme®'s coating, explaining the observed increases in T1. Thus diffusion-weighted imaging and MRI was used to obtain results emanating from the apparent diffusion coefficient map. It was found that the Flutaxl- and doxorubicin-loaded Feraheme® had lower diffusion coefficients than the unloaded Feraheme® (FIGS. 4C-D), showing that cargo intercalation (loading) obstructed the free diffusion of water molecules.

### EXAMPLE VI

Exemplary Demonstration of Decreased T2 Values and Increased T1 Values with Particle Aggregation.

Target-induced clustering of IONP was used in sensitive assays for the detection of numerous biomolecules and targets (Grimm, et al., Novel nanosensors for rapid analysis of telomerase activity. Cancer Res 2004, 64(2):639-43, Perez, et al., Integrated nanosensors to determine levels and functional activity of human telomerase. Neoplasia 2008, 10 (10): 1066-72; Perez, et al., Magnetic relaxation switches capable of sensing molecular interactions. Nat Biotechnol 2002, 20 (8): 816-20, each of which is herein incorporated by reference in its entirety). Specifically, it was demonstrated that the nanoparticles form extensive supramolecular assemblies in the presence of their target (Perez, et al., Magnetic relaxation Switches Capable of Sensing Molecular Interactions. Nat Biotechnol 2002, 20 (8), 816-20; Koh, et al., Nanoparticle-target Interactions Parallel Antibody-protein Interactions. Anal Chem 2009, 81 (9), 3618-22; Kaittanis, et al., The Assembly State between Magnetic Nanosensors and Their Targets Orchestrates Their Magnetic Relaxation Response. J Am Chem Soc 2011 , 133 (10), 3668-76, each ofwhich is herein incorporated by reference in its entirety). The formation of these assemblies that consisted of multiple nanoparticles was predominantly associated with T2 decreases and no reported effect on T1.

Since this result was different from the observed increases in T2 and T1 during cargo incorporation, T1, was measured for affects during Feraheme®'s aggregation. As a model target-induced clustering system, Concanavalin A (Con A), a protein that has high affinity towards carbohydrates, (Asian, et al., Nanogold-plasmon-resonance-based Glucose Sensing. Anal Biochem 2004, 330 (1), 145-55; Yoshizumi, et al., Self-assembled Monolayer of Sugar-carrying Polymer Chain: Sugar Balls from 2-methacryloyloxyethyl D-glucopyranoside. Langmuir 1999, 15 (2), 482-488, each of which is herein incorporated by reference in its entirety) was used to facilitate the clustering of Feraheme®, since it was coated with carboxymethyl dextran. Addition of the Con A ([Con A]_{fina}i=50 μg/mL) to Feraheme® ([Fe]=23 μg/mL) induced decrease in the solution's T2 but increase in the T1 (FIGS. 5A-B), with nanoparticle aggregation confirmed with DLS (FIG. 5C). Likewise, the differential effect on IONP's magnetic properties was reflected on their relaxivities, with the r₂ increasing and decreasing since they are inversely proportional to the relaxation times T2 and T1 respectively (FIGS. 13A-B). Moreover, when enhanced nanoparticle aggregation was facilitated in the presence of excess dextran ([Dextran]=2.5 mg/mL), marked increase in the T1 value was observed, with the T2 decreasing (FIGS. 5A-C) due to Feraheme®'s Con A-induced clustering. Taken together, these results demonstrate that IONP aggregation mediates significant decreases in the spin-spin relaxation times (T2), while promoting increases on T1, which become prominent as the aggregates become bigger. This may explain why previous studies did not identify the effect of nanoparticle clustering on the T1 process (Taktak, et al., Multiparameter Magnetic Relaxation Switch Assays. Anal Chem 2007, 79 (23), 8863-9, herein incorporated by reference in its entirety) as one contribution is the extent of the aggregation state. In summary, these results showed that cargo incorporation and the associated T2 and T1 increases were discerned from nanoparticle clustering and its effect on IONP magnetic properties, thus showing that sensitive characterization of diverse cargo-loaded formulations would be of use in research and clinical settings.

### EXAMPLE VII

Exemplary Demonstration of pH-Dependent Release of Cargo That Alterd the Magnetic Properties of Loaded IONP.

One application for intercalated drugs in coated IONPs is the use of IONP as a drug delivery platform, which allows for control of loading (and release) by changes in T2 and T1. Ideally, the cargo should be retained at physiological conditions and released in the presence of environmental cues, such as abnormally low pH. This feature is ideal for cancers that exhibit acidic interstitial pH, due to upregulated glycolysis as a result of signaling and metabolic alterations, collectively described as the Warburg effect (Vander Heiden,et al., Understanding the Warburg effect: the metabolic requirements of cell proliferation. Science 2009, 324 (5930), 1029-33, herein incorporated by reference in its entirety).

In order to examine the potential use of Feraheme® as a smart drug delivery system, the stability of drug-loaded Feraheme® in phosphate-buffered saline (PBS) at physiologic pH of 7.4 was investigated. Incubation of doxorubicin-loaded Feraheme® in pH-adjusted buffer for 24 hours did not reveal major changes in the T2, and T1, spanning the pH range encountered during physiological conditions (FIGS. 15A-B). Since doxorubicin is fluorescent and was used in spectroscopic and fluorescence microscopy studies (for example, see, Santra, et al., Cell-specific, Activatable, and Theranostic Prodrug for Dual-targeted Cancer Imaging and Therapy. J Am Chem Soc 201 1, 133 (41), 16680-8, herein incorporated by reference in its entirety), the nanoparticle's fluorescence emission was monitored across the pH range shown in FIG. 15C.

However there were significant changes in fluorescence emission intensity. Changes in T2 and T1 were observed at a lower pH of 7.0 thus further experiments were done to identify whether the cargo was being released upon acidification of the aquatic milieu. Employing a dialysis chamber to separate the nanoparticles from the potentially released drug, doxorubicin-carrying Feraheme® was incubated in IX PBS adjusted to pH 6.8 and 6.0. Rapid decreases in T2 and T1 were observed under these mildly acidic conditions (FIGS. 6A-B), which were accompanied with decreases in the nanoparticles' fluorescence emission (FIGS. 6C) due to release of doxorubicin as confirmed with HPLC-based spectrophotometry (FIGS. 6D). On the other hand, no changes in T2 and T1 were observed at pH 7.2, likely due to stable accommodation of doxorubicin within Feraheme®'s coating, which is supported by the preparation's unaltered fluorescence emission.

To further confirm that these changes were mediated by cargo release, DLS was performed prior and after incubation at these pH levels. Results indicated that the nanoparticle size and distribution were constant throughout the experiment, with the nanoparticles being stable after 2 h at pH 6.0 (FIG. 6E-F). Plausibly, release of cargo at acidic pH may have been facilitated due to disturbance of the weak electrostatic interactions that mediate the association of the drug with the nanoparticles' polymeric backbone secondary to hydrophobic interactions. It is possible that changes in the protonation of the polymer' s side chains may disturb hydrogen bonding and van der Waals interaction between the 10NP's coating and the cargo, thus triggering release at lower pH. This further demonstrates that the effect of cargo mtercalation within the shell of a coated IONP of the present inventions was reversible, since payload release facilitated recovery of their magnetic properties, that lead to decreased T2 and T1.

Therefore, Feraheme® is contemplated for use for delivery of chemotherapeutic cargo to the tumor, with the lesion's acidic pH serving as an endogenous trigger for rapid drug release at the tumor's vicinity, maximizing therapeutic efficacy.

### EXAMPLE VIII

Exemplary Demonstration of Effective Feraheme®-Based Delivery of Chemotherapeutics

After establishing that cargo release caused decreased T2 and T1, cargo-loaded IONPs were used in testing whether IONP would deliver therapeutic payloads within cancer cells. Serum stability measurements were taken for establishing stability of cargo loaded coated IONPs in vivo. Serum alone had a T2 value of 600±10 ms and T1 of 1700±30 ms, which remained unaltered during the course of the study. DiR-carrying Feraheme® was found to be stable for up to 8 days in sterile fetal bovine serum (FIGS. 15A-D), with no changes in the relaxation times. However, doxorubicin- and Flutaxl -loaded nanoparticles released their payload having different types kinetic profiles, as indicated by concomitant changes in the relaxation times (FIGS. 7A-D). Specifically, doxorubicin was completely released from it's IONP carrier within less than four days, which resulted in relaxation times that were comparable to those of the unloaded nanoparticles whose T2 and T1 remained constant throughout the study (FIGS. 7A-B). On the other hand, the Taxol® derivative Flutaxl demonstrated sustained release in serum over the ten-day duration of the study, where approximately 50% of the drug was released after 7 days (FIGS. 7C-F). These different release patterns were contemplated to be the result of different physical and chemical characteristics of each payload molecule.

For example, doxorubicin, a small anthracycline DNA intercalator (MW: 580), might be released faster, due to lower hydrogen bonding formation with the shell polymer that resulted an overall weaker association with the nanoparticles. On the other hand, Flutaxl, a fluorescent microtubule-stabilizing diterpene (MW: 1337), has multiple oxygen and nitrogen atoms would facilitate a stronger noncovalant association within the nanoparticle's polymeric coating molecule. Additionally, Flutaxl has multiple molecular structures (segments) that would favor multiple hydrophobic interactions with shell molecules. In fact, this latter observation is contemplated to contribute to the stabilization of DiR (MW: 1013) within the nanoparticle shells due to the presence of two eighteen-carbon-long aliphatic chains.

Further, the inventors' contemplated that IONPs having multiple types of molecular payloads would be able to target several oncogenic pathways instead of merely having one drug for one targeted interaction. Such a strategy was contemplated to increase the drugs' release at the tumor site while maximizing their circulation time, in a process that is orchestrated by the payload' s intrinsic characteristics and initiated by the tumor's aberrant glycolytic activity, without subjecting the drugs to modification.

Furthermore, since enhanced permeability and retention (EPR) was a feature of tumors, it was determined whether cancer cells, such as the prostate cancer cell LNCaP, would uptake the cargo-loaded Feraheme® thus facilitating the intracellular release of the cargo from the nanoparticles. Incubation of LNCaP with the Doxorubicin-loaded Feraheme® for 48 h resulted in significant nanoparticle uptake, as indicated by the enhanced fluorescence due to the presence of doxorubicin, which was determined through fluorescence microscopy (FIG. 8A). In addition to determining whether LNCaP cells would uptake doxorubicin-loaded Feraheme®, further studies investigated whether prostate cancer cells would uptake Feraheme® that carried larger cargo (i.e. larger molecules), such as the near-infrared fluorophore DiR. After 48 h incubation with the loaded nanoparticles, the cells were washed, trypsinized and the resulting cell pellets were resuspended in IX PBS pH 7.4. Quantification of the cell-associated DiR fluorescence was achieved through the Odyssey imager, which demonstrated high fluorescence emission at 800 nm excitation in cells treated with the DiR-carrying Feraheme® (FIGS. 8B).

Additionally, experimental results obtained during the development of the present inventions showed that in addition to nanoparticle uptake, the unloading of DiR within the cells partially restored Feraheme®'s magnetic properties, which approached those of the original pre-loaded state. The r₂ and τ_{\} relaxivities of the unloading nanoparticles (where intercalated cargo molecules were disassociating from noncovlant bonds with coating molecules) were higher than those of the loaded Feraheme® (FIGS. 8C-D). Taken together, these findings show that the effects of the cargo on Feraheme®'s magnetic properties were reversible, since payload intercalation relied on non-covalent bonding between shell molecules and loaded molecules. Thus, this may facilitate the monitoring of drug loading on Feraheme® and release via magnetic resonance methods, for in vitro and in vivo applications. Particularly for drugs with poor aqueous solubility, this approach can significantly improve their circulation time and stability with a clinically approved nanoparticle-based delivery system. Since the intercalation process was reversible and does not modify the drug chemically, these drugs retain their structure—function mode of action, exerting their biological effect once released at the acidic tumoric bed or within cancer cells, upon uptake through endocytic processes. Findings during the development of the present inventions described herein, demonstrated that magnetic relaxation can be used as an attractive method for the sensitive characterization of non-fluorescent payloads carried by (superparamagnetic nanoparticles in pharmacological and clinical studies, utilizing portable relaxometers, (Taktak, et al., Multiparameter Magnetic Relaxation Switch Assays. Anal Chem. (2007) 79(23):8863-9; Issadore, et al., Miniature Magnetic Resonance System for Point-of-care Diagnostics. Lab Chip (2011) 11 (13):2282-7, each of which is herein incorporated by reference in its entirety). MR analyzers and MRI and expanding the applications of IONP in new areas, such as chemoprevention and tumor-directed, personalized therapy, such as cargo-loaded iron oxide nanoparticles were tested on cancer cells in order to measure the release of their cargo intracellularly. Control studies demonstrated absence of cytotoxicity in cells treated with unloaded Feraheme®, in accordance with other iron oxide nanoparticle formulations that have different polymeric coatings than this clinically approved agent. Therefore, the in vitro toxicity of Feraheme® carrying chemotherapeutic agents was investigated. Surprisingly, treatment of human prostate adenocarcinoma cells (LNCaP) with drug-carrying Feraheme® resulted in significant cell death, which was more pronounced than treatment with equimolar concentration of the free drug (FIG. 9A). For instance, doxorubicin-loaded Feraheme® induced more than 75% reduction in cell viability, as opposed to doxorubicin alone that caused 60% decrease. Similarly, Flutaxl -carrying nanoparticles were 10%) more cytotoxic than the nascent drug, further supporting the notion that delivery of chemotherapeutics with this nanoplatform does not compromise the drug's cytotoxic activity, due to the absence of covalent modifications during the intercalation process. Apart from conventional chemotherapeutics that target DNA synthesis and mitosis, anticancer drugs that target enzymes involved in signal transduction and cholesterol/sterol biosynthesis were tested. These results indicated that the loaded-Feraheme® preparations were more effective than free Alendronate and BKM120 (FIG. 9B), likely due to the improved aqueous stability and bioavailability that the nanoparticles confer to their cargo.

### EXAMPLE IX

Exemplary Demonstration of Effective Treatment With Duel Loaded IONP.

Because certain cancers, such as prostate cancer, have more than one pathway involved in oncogenesis and metastasis, Feraheme® was utilized as a drug delivery vehicle for combinatorial therapy. Specifically, since in prostate cancer the crosstalk between the androgen receptor pathway and the PI3K cascade leads to resistance to agents targeting one of the two pathways, (Carver, et al., Reciprocal feedback regulation of PI3K and androgen receptor signaling in PTEN-deficient prostate cancer. Cancer Cell (201 1), 19 (5), 575-86), herein incorporated by reference in its entirety), developing a strategy that targeted both pathways would be ideal, leading to improved therapeutic efficacy.

Thus, BEZ235, a PI3K inhibitor, and the androgen receptor antagonist MDV3100 were intercalated together into the outer coating of the same IONP, in order to deliver both drugs within prostate cancer cells. The prostate cancer cell line LNCaP was treated with this duel loaded IONP. A prostate cancer cell line LNCaP, which has a functional AR cascade, showed that Feraheme® carrying both BEZ235 and MDV3100 led to more than 30% reduction in cell viability, as opposed to cells treated with one free drug or a combination of both free drugs (FIG. 9C).

In vivo studies revealed that doxorubicin-carrying Feraheme® is more effective than free doxorubicin, after three iv injections of either free or intercalated doxorubicin (FIG. 9D). Specifically, the mice were treated with equal concentrations of the drug on day 0, 2 and 6, and tumor growth was determined with microcalipers. Control animals were treated with 100 iL of 10% DMSO-containing PBS to match the DMSO content of the free doxorubicin injection, since the drug was dissolved in DMSO and diluted to the desired concentration in PBS. On day 6 and 8, the tumors were measured, and the change in tumor growth was determined by comparing the volume of tumors from treated animals with those of control ones. Surprisingly, doxorubicin-loaded Feraheme® IONPs were able to achieve 10% tumor regression after two injections, as opposed to some tumor growth after treatment with free doxorubicin. In fact, after three injections, when the tumors were measured on day 8 for each of these treatments, the drug-loaded Feraheme® was able to suppress tumor growth by almost 50% as opposed to 34% suppression from the use of free drug. These measurements showed that treatment with an ampipathic agent that was intercalated within Feraheme® enhanced its bioavailability. This enhancement was contemplated to be the result of the loaded coated Feraheme®'s capability to specifically deliver drug within tumors at effective dosages while preventing association of drug with proteins and lipids in solution. Whereas nonspecific association of free drug with proteins and lipids in solution reduced the effective dosage, i.e. the amount of administered drug compared with effect.

### EXAMPLE IX

These results show additional exemplary drug loading on Feraheme® IONPs and effects after treating cells in vitro.

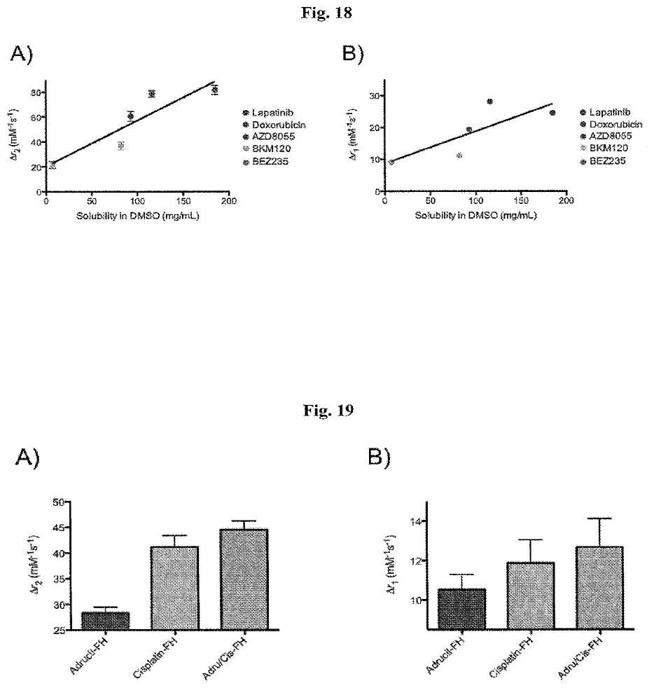

Single-drug nanoparticles were made by using Feraheme® IONPs. Feraheme® IONPs were not magnetically separated. Individual drugs used for loading included Lapatnib, Doxorubicin, AZD8055, BKM120, and BEZ235. Magnetic properties of the loaded IONPs were measured for both r_{\} (mM″¹s) and r₂ (mM′V¹) in relation to solubility in DMSO (mg/ml), see FIG. 18. Results showed relatively values from lowest to highest, BEZ235, BKM120, AZD8055, Doxorubicin and Lapatnib for solubility in DMSO. While change in r₂ relaxivities showed values from low to high change for r, BEZ235, BKM120, AZD8055, with similar values for Doxorubicin and Lapatnib. R\ changes showed, from lowest to highest, BEZ235 and BKM120, AZD8055, Lapatnib then Doxorubicin. In general, low solubility in DMSO produced lower changes in r₂ and rj while higher solubility in DMSO produced greater changes in magnetic properties.

Drug loaded nanoparticles were also made by using Feraheme® IONPs and loading therapeutics, Adrucil and Cisplatin. Single and double loaded IONPs were made by individually loading Adrucil and Cisplatin at a final concentration of 50 uM (uM: micromolar). Double-drug nanoparticles were co-loaded with Adrucil and Cisplatin which had a concentration of 25 uM of each drug. Feraheme® IONPs were not magnetically separated. Magnetic properties of the loaded IONPs were measured as changes compared to IONPs without loaded drugs, see FIG. 19. Loaded IONPs showed increased r₂ (niMA\hat{ }s⁻¹) (FIG. 19A) and r_{\} (mM′V¹) (FIG. 19B) values over IONPs without drugs.

Therapeutic loaded coated Feraheme® IONPs were used for treating cells in vitro. Human prostate adenocarcinoma cells LNCaP (LNCaP-wt) cells were seeded at a density of 25,600 cells in culture wells, while androgen-receptor-overexpressing LNCaP prostate cancer cells (LNCAP-AR) cells were seeded at 14,000 cells per well, and grown at 37° C., 5% CO₂ for 48 hours. Afterwards, the cells were treated with 10 uL of equimolar amounts (50 uM) of either free drug(s) or the nanoparticles for 48 h. Cell viability was assessed via the Alamar Blue method.

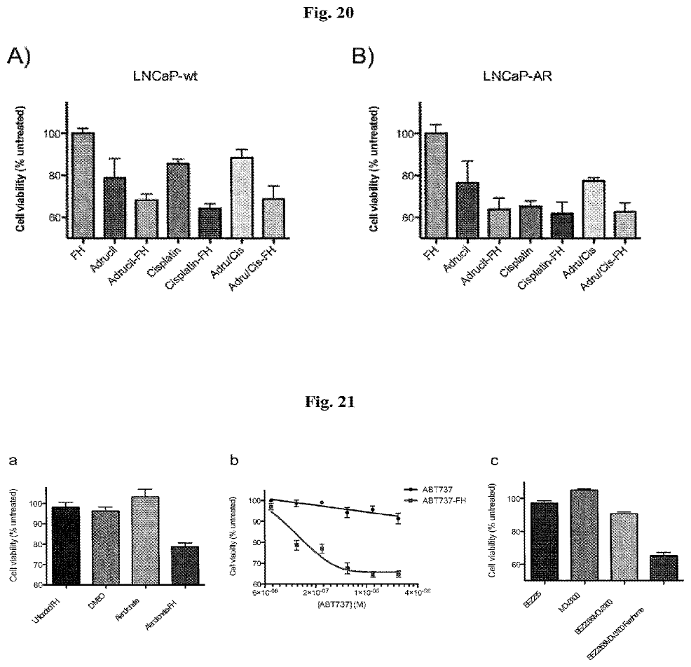

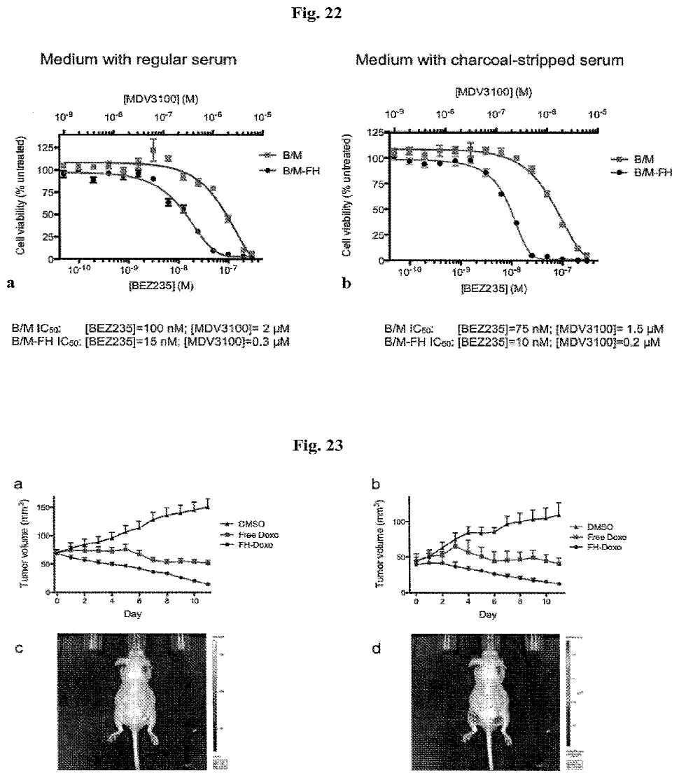

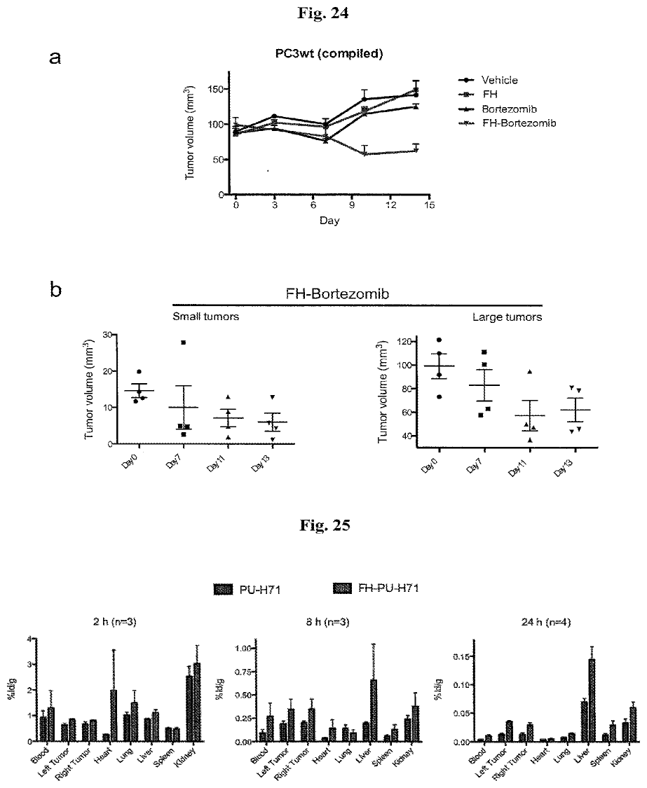

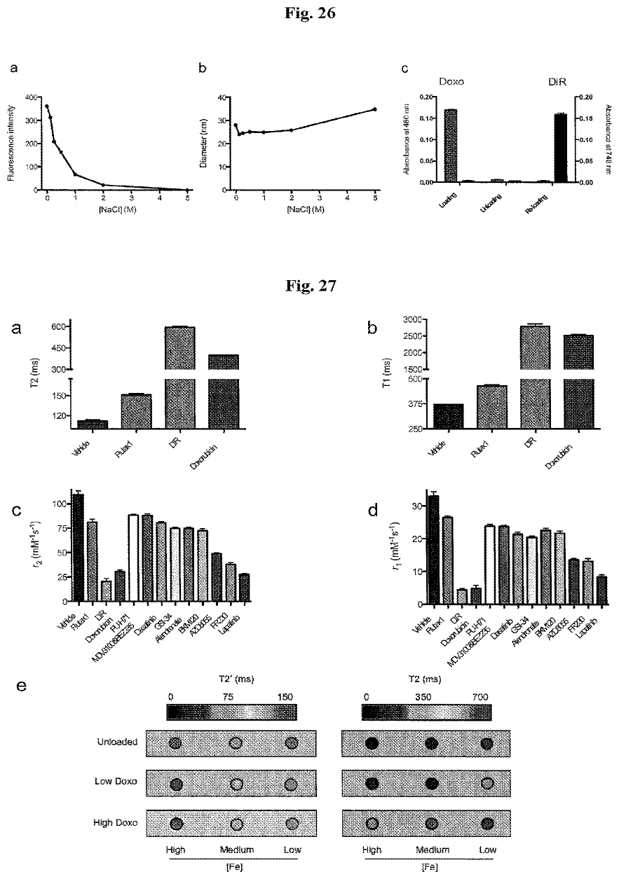

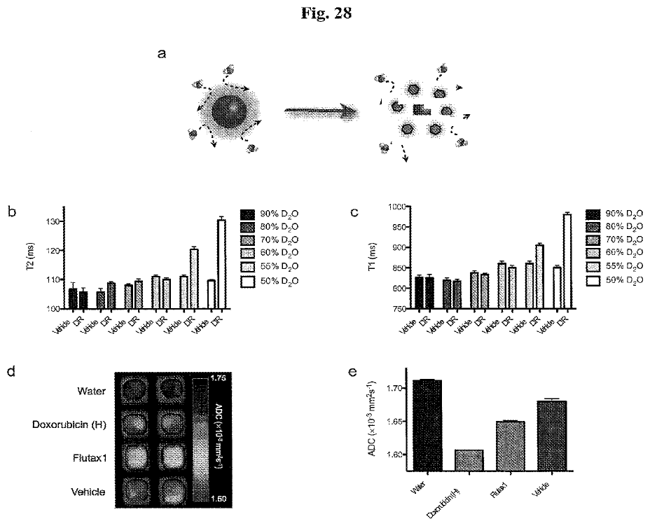

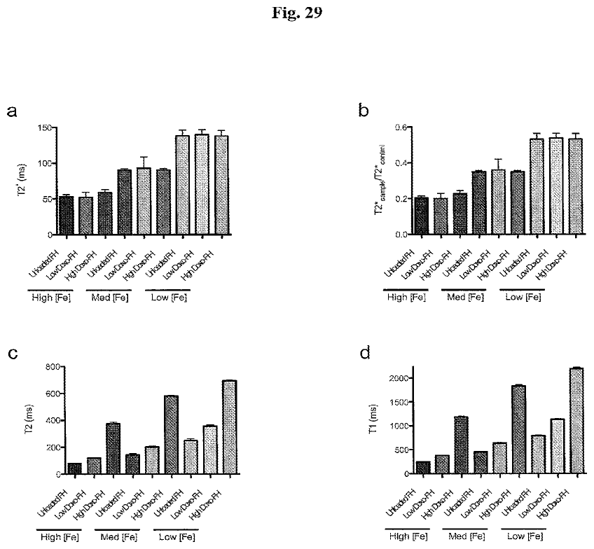

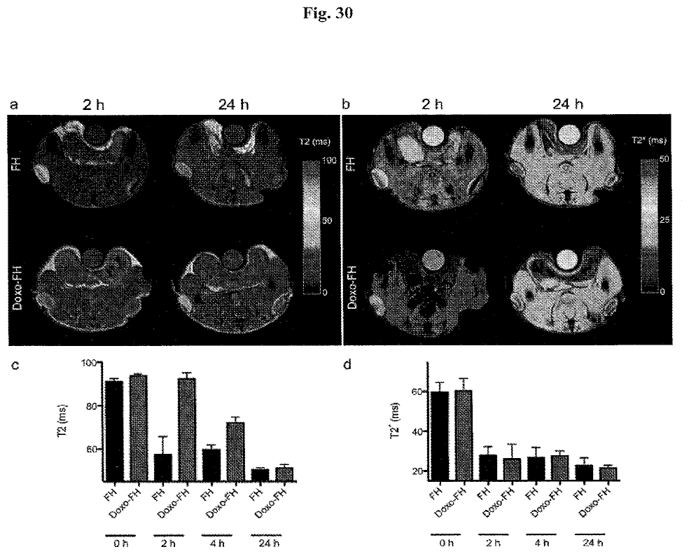

LNCaP-wt cells showed a greater susceptibility to drugs loaded onto FH than free drugs, FIG. 20A. LNCAP-AR cells showed mixed results with Adrucil-FH and Adrucil (Adru)/Cisplantin (Cis)-FH more effective than free drugs while Cispatin showed similar levels in loss of cell viability, FIG. 20B. Surprisingly, LNCAP-AR cells were more susceptible than LNCaP-wt cells to Adrucil (Adru)/Cisplantin (Cis)-FH.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in cellular biology, cancer cell biology, biochemistry, chemistry, organic synthesis, imaging diagnostics or related fields are intended to be within the scope of the following claims.

What is claimed is:

1. A method of loading, the method comprising
a) providing
i) a coated (super)paramagnetic iron oxide nanoparticle core, wherein said coated iron oxide nanoparticle core has a coat comprising at least one molecule selected from the group consisting of a poly(acrylic acid), carboxymethyl dextran, and polyglucose sorbitol carboxymethylether; and
ii) a cargo molecule capable of being attached to said coat;
b) adding said cargo molecule solution dropwise without inducing precipitation to said nanoparticle core; and
c) mixing said cargo molecule with said nanoparticle core under conditions such that said cargo molecule non-covalently attaches to said coat.

2. The method of claim 1, further comprising a magnetic device for obtaining T1 and T2 of said (super)pararnagnetic iron oxide nanoparticles.

3. The method of claim 2, wherein said T1 and T2 increase as said cargo molecule is attached to said coat.

4. A method of delivering a therapeutic to a cell, comprising
a) providing
i) 1 drug delivery composition, comprising, a (super)paramagnetic iron oxide nanoparticle core, wherein said nanoparticle core comprises a coat non-covalently attached to a therapeutic; and said coat comprises at least one molecule selected from the group consisting of poly(acrylic acid), carboxymethyl dextran, and polyglucose sorbitol carboxymethylether; and
ii) a cancer cell; and
b) administering said composition to said cell, under conditions such that the cancer cell undergoes cell death.

5. The method of claim 4, wherein said cancer cell is a prostate cancer cell.

6. The method of claim 4, wherein said cancer cell is a tumor cell.

7. The method of claim 4, further providing a magnetic device for obtaining T1 and T2 of said (super)pararnagnetic iron oxide nanoparticles.

8. The method of claim 7, wherein said method further comprises the step of using said device for obtaining T1 and T2 of therapeutic loaded coated (super)paramagnetic iron oxide core nanoparticles before administration to said cell.

9. The method of claim 7, wherein step b) further comprises using said device for obtaining T1 and T2 as said therapeutic is administered.

10. The method of claim 9, wherein said T1 and T2 of said nanoparticles is decreased in relation to T1 and T2 obtained before administration.

11. The method of claim 4, wherein said cell is located in a patient.

12. The method of claim 9, wherein said a magnetic device is a magnetic resonance imaging device.

13. The method of claim 1, wherein said coat further comprises at least one amine-functionalized molecule.
